(19) United States
(12) Patent Application Publication
(10) Pub. No.: US 2023/0355672 A1
(43) Pub. Date: Nov. 9, 2023

(54) HUMANIZED ANTIGEN BINDING UNITS FOR TARGETING OSTEOSARCOMA CELLS

(71) Applicant: Oslo Universitetssykehus HF, Nydalen (NO)

(72) Inventors: Sébastian Wälchli, Oslo (NO); Øyvind S. Bruland, Oslo (NO); Darragh McCann, Magherfelt (IE); Samantha Crawford, Randalstown (GB); Else Marit Inderberg, Oslo (NO); Monica Berrondo, Austin, TX (US); Susana Kaufmann, Austin, TX (US); Jacob Byerly, Austin, TX (US)

(21) Appl. No.: 18/011,404

(22) PCT Filed: Jun. 24, 2021
(86) PCT No.: PCT/EP2021/067376

§ 371 Date: Dec. 19, 2022

Related U.S. Application Data
(60) Provisional application No. 63/043,503, filed on Jun. 24, 2020.

(30) Foreign Application Priority Data
Jun. 24, 2020 (NO) 20200740
Jun. 24, 2020 (NO) 20200741

(51) Int. Cl. A61K 35/17; C07K 16/30; A61P 35/00

(52) U.S. Cl. CPC A61K 35/17; C07K 16/30; A61P 35/00; A61K 2039/5158

(57) ABSTRACT

Provided herein are proteins comprising a novel antigen binding domain. Said antigen binding domain, and proteins comprising it, are able to bind to osteosarcoma cells under physiological conditions. The antigen binding domain comprises an antibody light chain variable domain (VL) and an antibody heavy chain variable domain (VH), each comprising three complementarity determining regions (CDRs) flanked by human framework sequences. The human framework sequences reduces the risk of triggering unwanted immunogenic responses against the antigen binding domain. This may be especially important in a therapeutic setting wherein a drug is administered repeatedly to a human osteosarcoma patient.

### CROSS REFERENCE TO RELATED APPLICATIONS

This application is a National Stage application of PCT/EP2021/067376, filed Jun. 24, 2021, which claims priority to Norwegian Patent Application No. 20200740, filed Jun. 24, 2020, Norwegian Patent Application No. 20200741, filed Jun. 24, 2020, and U.S. Provisional Application 63/043,503, filed Jun. 24, 2020, all of which are incorporated by reference in their entirety herein.

### SEQUENCE LISTING

The Instant Application contains a Sequence Listing which has been submitted electronically in ASCII format and is hereby incorporated by reference in its entirety. Said ASCII copy, created on Jul. 14, 2023 is named “OSA0053US2_ST25” and is 195,698 bytes in size.

### TECHNICAL FIELD OF THE INVENTION

The invention is related to the field of cancer therapy. In particular, it relates to novel targeting units and chimeric antigen receptors (CARs) comprising them, nucleic acids encoding the targeting units, nucleic acids encoding the CARs, immune cells expressing CARs and their utility in medicine for treatment of osteosarcoma.

### BACKGROUND

Osteo sarcoma (OS) is the most common primary bone cancer and the eighth most common form of cancer among children and adolescents.

One clinical trial concerning sarcoma patients including osteosarcoma patients has been reported based on targeting of HER2 by autologous T cells expressing a CAR (Ahmed et al. 2015, J Clin Oncol. 2015 May 20; 33(15):1688-96). In this dose-escalation study, the clinical benefit of HER2-CAR T cell infusion was clearly limited, indicating that further manipulation of the immune system will be essential for worthwhile benefits to be obtained.

In order to achieve a therapeutic CAR T cell, the cell needs to express the CAR in a sufficient amount in the cell membrane, and the antigen binding domain has to convey sufficient affinity and specificity in the CAR construct. It can be expected that only a fraction of CAR T cells with in vitro activity will successfully migrate to tumor metastases in vivo and infiltrate the hostile tumor microenvironment of a solid tumor like OS. Furthermore, the CAR T cells will likely need to sustain their activity over time in order to provide a therapeutic effect in vivo. It is therefore not trivial, but very desirable to obtain novel CARs able to provide a therapeutic effect on OS in vivo when expressed in the cell membrane of immune cells.

Five drug products based on CARs are currently approved:
Kymriah® (tisagenlecleucel),
Yescarta® (axicabtagene ciloleucel),
Tecartus® (brexucabtagene autoleucel),
Breyanzi® (lisocabtagene maraleucel) and
Abecma® (idecabtagene vicleucel)

According to the FDA approved package inserts, all these CARs comprise a murine scFv.

However, murine sequences may trigger unwanted immunogenicity against the therapeutic cells during therapy in humans. Successful humanization of murine antibodies has been reported, but in the context of CAR therapy, retained specificity and affinity for the target antigen is utterly important.

### SUMMARY OF THE INVENTION

Provided herein are proteins comprising a novel antigen binding domain. Said antigen binding domain, and proteins comprising it, are able to bind to osteosarcoma cells under physiological conditions. The antigen binding domain comprises an antibody light chain variable domain (VL) and an antibody heavy chain variable domain (VH), each comprising three complementarity determining regions (CDRs) flanked by human framework sequences. The human framework sequences reduces the risk of triggering unwanted immunogenic responses against the antigen binding domain. This may be especially important in a therapeutic setting wherein a drug is administered repeatedly to a human osteosarcoma patient.

As demonstrated in Example 2 and visualized in FIGS. 4 and 5, it is found that T cells expressing CARs comprising humanized scFv's derived from the TP1 Fv, have increased activity in a cytokine-assay involving osteosarcoma cells relative to T cells expressing CARs comprising the murine Fv.

It is also found that T cells expressing CARs comprising humanized scFv's derived from the TP3 Fv, have retained activity in a cytokine-assay involving osteosarcoma cells relative to T cells expressing CARs comprising the murine Fv. The latter may be beneficial because CARs comprising the murine Fv have already been demonstrated to work well in vivo.

Interestingly, when tested in a cytotoxic assay (FIG. 5), the same humanized CARs expressed in primary human T cells were either very similar to the original murine construct (OSCAR-1) or a bit weaker (OSCAR-3). It demonstrates that structure-prediction algorithms are limited in predicting the impact of the modifications on the cellular outcome of the humanized CAR.

In relation to CAR therapy, transient expression of CARs via mRNA may be an attractive option due to the inherent risk of off-target effects and on-target off-tumor effects from CAR T cells. However, the transient expression may require more frequent administration of the therapeutic cells in order to achieve a therapeutic effect. During such repeated treatment, the CARs herein may be particularly useful

In one aspect the present invention provides a protein for targeting of osteosarcoma cells,
comprising a VL and a VH which together form an antigen binding unit,
wherein the VL comprises three complementarity determining regions (CDRs); CDR1, CDR2 and CDR3 which respectively are represented by the amino acid sequences SEQ ID NO: 1, WAS, and SEQ ID NO: 2
and
wherein the VH comprises three CDRs; CDR1, CDR2 and CDR3 which respectively are represented by the amino acid sequences SEQ ID NOs: 3, 4 and 5,
and
wherein all the CDRs are flanked by human framework sequences, and optionally wherein each of the human framework sequences, independently, comprises 0 to 5 amino acid substitutions.

In one embodiment of the first aspect the VL is selected from the group consisting of VL1A (SEQ ID NO: 11), VL1B (SEQ ID NO: 12), VL1C (SEQ ID NO: 13), VL1D (SEQ ID NO: 14), VL1E (SEQ ID NO: 15), VL1F (SEQ ID NO: 16), VL1G (SEQ ID NO: 17) and VL1H (SEQ ID NO: 18), and the VH is selected from the group consisting of VH1A (SEQ ID NO: 19), VH1B (SEQ ID NO: 20) and VH1C (SEQ ID NO: 21), VH1D (SEQ ID NO: 22), VH1E (SEQ ID NO: 23), VH1F (SEQ ID NO: 24), VH1G (SEQ ID NO: 25) and VHIH (SEQ ID NO: 27).

In one embodiment of the first aspect the antigen binding unit is selected from the scFv's represented by SEQ ID NO: 43 to SEQ ID NO: 76.

In a second aspect the present invention provides a Chimeric Antigen Receptor (CAR) comprising the protein according to the first aspect and any embodiments thereof.

In one embodiment of the second aspect the CAR according comprising a human CD8α a hinge.

In a third aspect the present invention provides a nucleic acid encoding the protein according to the first aspect or the CAR according to the second aspects.

In a fourth aspect the present invention provides an immune cell expressing a CAR according according to the second aspect in its cell membrane.

In a fifth aspect the present invention provides a pharmaceutical composition comprising a protein according to the first aspects and embodiments therein.

In one aspect the present invention provides a pharmaceutical composition comprising a cytotoxic immune cell according to the fourth aspect.

In a sixth aspect the present invention provides a method of treatment of osteosarcoma in a human patient comprising the step of administering a pharmaceutical composition according to the fifth aspect.

In a seventh aspect the present invention provides an antibody comprising the protein according to the first aspect and embodiments therein.

In a eighth aspect the present invention provides a pharmaceutical composition comprising the antibody according to the seventh aspect for use in treatment of osteosarcoma.

In a ninth aspect the present invention provides method for treatment of osteosarcoma in a human patient comprising the step of administering a pharmaceutical composition according to the eighth aspect.

In a tenth aspect the present invention provides an antibody according to the seventh aspect for use in an in vitro diagnostic method.

In an eleventh aspect the present invention provides a protein for targeting of osteosarcoma cells,
comprising a VL and a VH which together form an antigen binding unit,
wherein the VL comprises three complementarity determining regions (CDRs); CDR1, CDR2 and CDR3 which respectively are represented by the amino acid sequences SEQ ID NO: 6, LAS and SEQ ID NO: 7
and
wherein the VH comprises three CDRs; CDR1, CDR2 and CDR3 which respectively are represented by the amino acid sequences SEQ ID NOs: 8, 9 and 10.

In one embodiment of the eleventh aspect the VL is selected from the group consisting of VL3A (SEQ ID NO: 27), VL3B (SEQ ID NO: 28) and VL3C (SEQ ID NO: 29), VL3D (SEQ ID NO: 30), VL3E (SEQ ID NO: 31), VL3F (SEQ ID NO: 32), VL3G (SEQ ID NO: 33), VL3H (SEQ ID NO: 34) and the VH is selected from the group consisting of VH3A (SEQ ID NO: 35), VH3B (SEQ ID NO: 36) and VH3C (SEQ ID NO: 37), VH3D (SEQ ID NO: 38), VH3E (SEQ ID NO: 38), VH3F (SEQ ID NO: 40), VH3G (SEQ ID NO: 41), and VH3H (SEQ ID NO: 42).

In one embodiment of the eleventh aspect the antigen binding unit is selected from the scFv's represented by SEQ ID NO: 77 to SEQ ID NO: 110.

In a twelfth aspect the present invention provides a Chimeric Antigen Receptor (CAR) comprising the protein according to the eleventh aspect and any embodiments thereof.

In one embodiment of the twelfth aspect the CAR according comprising a human CD8α a hinge.

In a thirteenth aspect the present invention provides a nucleic acid encoding the protein according to the eleventh aspect or the CAR according to the twelfth aspects.

In a fourteenth aspect the present invention provides an immune cell expressing a CAR according according to the twelfth aspect in its cell membrane.

In a fifteenth aspect the present invention provides a pharmaceutical composition comprising a protein according to the eleventh aspects and embodiments therein.

In one aspect the pharmaceutical composition comprising a cytotoxic immune cell according to the fourteenth aspect.

In a sixteenth aspect the present invention provides a method of treatment of osteosarcoma in a human patient comprising the step of administering a pharmaceutical composition according to the above aspect.

In a seventeenth aspect the present invention provides an antibody comprising the protein according to the eleventh aspect and embodiments therein.

In a eighteenth aspect the present invention provides a pharmaceutical composition comprising the antibody according to the seventeenth aspect for use in treatment of osteosarcoma.

In a nineteenth aspect the present invention provides method for treatment of osteosarcoma in a human patient comprising the step of administering a pharmaceutical composition according to the eighteenth aspect.

In one more aspect the present invention provides an antibody according to the seventeenth aspect for use in an in vitro diagnostic method.

### BRIEF DESCRIPTION OF THE DRAWINGS

FIG. 1 visualizes the process of humanization of a second-generation CAR which initially contained a murine scFv. From left to right: murine scFv-CD8α hinge-CD8α transmembrane domain-4-1BB co stimulatory domain-CD3 signaling domain.

FIG. 2A depicts substitutions in positions relative to the native human framework sequence for VH1A, VH1B and VH1C. Mutations from native are represented by an X above the mutant sequence.

FIG. 2B depicts substitutions in positions relative to the native human framework sequence for VL1A, VL1B and VL1C. Mutations from native are represented by an X above the mutant sequence.

FIG. 2C depicts substitutions in positions relative to the native human framework sequence for VH3A, VH3B and VH3C. Mutations from native are represented by an X above the mutant sequence.

FIG. 2D depicts substitutions in positions relative to the native human framework sequence for VL3A, VL3B and VL3C. Mutations from native are represented by an X above the mutant sequence.

FIG. 3A Visualization of the parent scFv comprising SEQ ID NO: 113 and SEQ ID NO: 114 with the murine CDRs coloured dark blue (1), light blue (2), dark green (3), light green (4), yellow (5) and red (6). The murine framework sequences are coloured purple (7).

FIG. 3B Overlay of the 25 novel scFv's (SEQ ID NO: 45 to SEQ ID NO: 78) wherein the human framework sequences are coloured gray (8) and with the murine CDRs coloured dark blue (1), light blue (2), dark green (3), light green (4), yellow (5) and red (6).

FIG. 4 In vitro CAR-dependent T cell stimulation by target cell lines was demonstrated by incubating the T cell line, Jurkat-76 constitutively expressing the reporter NFAT-GFP, co-expressing the indicated CAR constructs with osteosarcoma cell line (OSA). Clone 1326 (TP1 scFv-CD8α hinge-CD8α transmembrane domain-4-1BB costimulatory domain-CD3ζ signaling domain=OSCAR-1) and clone 1327 (TP3 scFv-CD8α hinge-CD8α transmembrane domain-4-1BB costimulatory domain-CD3ζ signaling domain=OSCAR-3) are second generation CARs comprising murine VL and VH sequences. Clone 1668, 1669, 1670 and 1671 are CARs with the same structure, but containing humanized scFv's derived from TP1, while clone 1672, 1673, 1674 and 1675 are CARs with the same structure, but containing humanized scFv's derived from TP3. PMA/ionomycin is a positive control for maximal stimulation. No target is J76-NFAT-CAR without target cells which sets the background levels. In this system, when J76-NFAT-GFP are stimulated through the T cell Receptor (TCR) signaling pathway (which is mimicked by the CAR), the NFAT promoter is activated and GFP is produced. The accumulation of GFP in the cytosol can be detected by fluorescent analysis (flow cytometry) and quantified. Here the GFP signal was quantified after 24 hours.

FIG. 5a-d: killing assay wherein CAR-expressing human primary T cells or mock T cells (control, without CAR) were co-incubated with ALPL negative (BL41) or ALPL positive (OSA) cells at the indicated E:T ratio for 4 hours (OSCAR-1) or 8 hours (OSCAR-3). The killing was monitored by BLI assay where the level of luciferase expressed by the target cells was detected and use to quantify the specific killing. As shown BL41 are not killed by T cells expressing OSCAR-1 or OSCAR-3 (a, b), whereas CAR-expressing T cells are able to lyse OSA cells (c, d). Terminology as in FIG. 4.

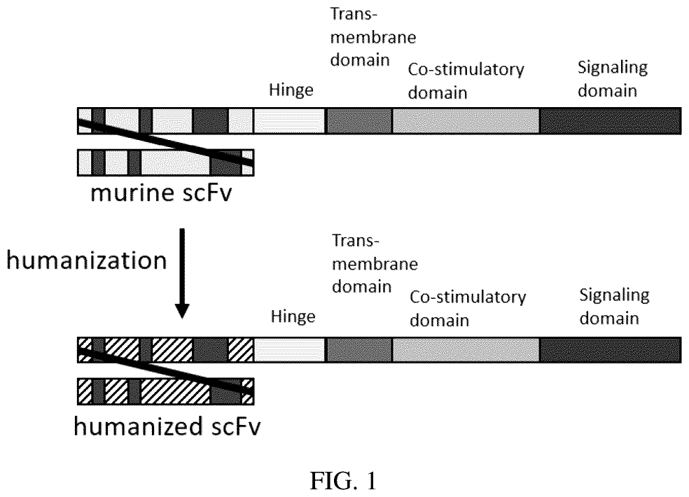

### DETAILED DESCRIPTION

Proteins comprising the novel antigen binding domains may have any suitable format including antibodies, scFv's, Fab's, immunotoxins, immunoconjugates, bispecific antibodies, CARs etc. Such proteins, especially antibodies, may be used to target osteosarcoma cells as such, and they may carry a toxic payload in the form of radioactive isotopes like ¹⁷⁷Lu, ²²⁴Ra or ²²⁵Ac.

In some embodiments proteins comprising the novel antigen binding domains described herein have cytotoxic effect on osteosarcoma cells.

As used herein, an antigen binding domain is a protein moiety able to bind an extracellular target epitope under physiological conditions, in particular physiological conditions in a tumor environment. The antigen binding domain comprises an antibody light chain variable domain (VL) and an antibody heavy chain variable domain (VH). Such variable domains are well-known for skilled persons.

The VH and VL may be connected by a disulfide bridges or a peptide linker. Alternatively, the two chains may be embedded in a Fab-fragment of an antibody or an antibody as such. In one embodiment, the antigen binding domain comprises or consists of VL-linker-VH. In another embodiment, the antigen binding domain comprises or consists of VH-linker-VL. Such antigen binding domains are often referred to as single chain Fv-fragments (scFv's). The linker has to have a certain length in order to allow the VH and VL to form a functional antigen binding domain. In one embodiment, the linker comprises 10 to 30 amino acid residues. In one embodiment, the linker comprises 15 to 25 glycine and/or serine residues.

Each VL and VH herein comprises three complementarity determining regions (CDRs) flanked by human framework sequences. Human framework sequences are structurally conserved regions that normally tend to form a β-sheet structure delicately positioning the CDRs for specific binding to the target antigen under physiological conditions. Many human framework sequences are available from known human antibodies and from the the international ImMunoGeneTics information system (IMGT) online database (see Giudicelli et al, Nucleic Acids Research, 2006, Vol. 34, Database issue D781—D784), but the term also covers human framework sequences comprising amino acid substitutions. Each of the human framework sequences may optionally comprise 0 to 15 amino acid substitutions. An amino acid substitution is a sequence wherein an amino acid residue in a specific position is substituted for a different amino acid residue at the corresponding position, apparent when the sequences are aligned. Each of the human framework sequences may optionally comprise 1 amino acid substitution. Each of the human framework sequences may optionally comprise 2 amino acid substitutions. Each of the human framework sequences may optionally comprise 3 amino acid substitutions. Each of the human framework sequences may optionally comprise 4 amino acid substitutions. Each of the human framework sequences may optionally comprise 5 amino acid substitutions. The substitutions may be conservative substitutions. Even if such framework sequences are not necessarily previously known from human antibodies, they may provide lower immunogenic risk compared to a murine framework sequence.

In one embodiment, 0 to 5 amino acid residues in the human framework sequences are substituted with the corresponding amino acid residue(s) from the murine parent sequences (SEQ ID NO: 111 and SEQ ID NO: 112). In one embodiment, human framework sequences are optionally substituted with the corresponding amino acid residue from the murine parent sequences (SEQ ID NO: 111 and SEQ ID NO: 112) at vernier positions.

In one embodiment, 0 to 5 amino acid residues in the human framework sequences are substituted with the corresponding amino acid residue(s) from the murine parent sequences (SEQ ID NO: 113 and SEQ ID NO: 114). In one embodiment, human framework sequences are optionally substituted with the corresponding amino acid residue from the murine parent sequences (SEQ ID NO: 113 and SEQ ID NO: 114) at vernier positions.

According to Safdari et al, Biotechnology and Genetic Engineering Reviews Volume 29, 2013—Issue 2:
The vernier positions in VH Framework1 are residues 2, 27, 28, 29 and 30.
The vernier positions in VH Framework2 are residues 47,48 and 49.
The vernier positions in VH Framework3 are residues 67, 69, 71, 73, 78, 93 and 94.
The vernier position in VH Framework4 is residue 103.
The vernier positions in VL Framework1 are residues 2 and 4.
The vernier positions in VL Framework2 are residues 35, 36, 46, 47, 48 and 49.
The vernier position in VL Framework3 is residue 71.
The vernier position in VL Framework4 is residue 98.

Collectively, scFv's comprising CDRs from a murine antibody and human framework sequences which each may optionally comprise 0 to 5 substitutions, are referred to as humanized scFv's. In some embodiments, substitutions may be back to the parent murine amino acid residue (also known as “back mutations”).

Accordingly, each of the framework sequenses in the VLs herein may, independently, contain 0 to 5 substitutions at the same number of vernier positions.

Accordingly, each of the framework sequenses in the VHs herein may, independently, contain 0 to 5 substitutions at the same number of vernier positions.

Accordingly, each of the framework sequenses in the scFv' herein may, independently, contain 0 to 5 substitutions at the same number of vernier positions.

In one embodiment, the human framework sequences are mature human framework sequences available from known human antibodies. Without being bound by theory, such framework sequences may convey very low risk of triggering unwanted immunogenic responses against the antigen binding domain, and at the same time increase the the likelihood of obtaining stable binding units which are expressed well in cellular systems.

The Framework1 sequence is N-terminal to the CDR1, Framework2 sequence is located between CDR1 and CDR2, while Framework3 sequence is located between CDR2 and CDR3.

Accordingly, both a VL and VH can be roughly visualized as follows, with the CDRs boxed and the N-terminal indicated as N—:

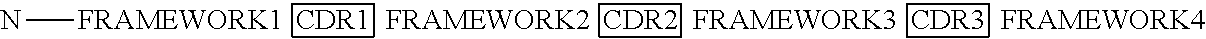

We provide eight examples of VL TP1 sequences comprising human framework sequences:

VL's TP1:

VL1A
(SEQ ID NO: 11)
DIVMTQSPSFLSASVGDRVTITCKASQGVGSAIAWYQQKPGQSPKLLIYWASIRHTGVP
DRFTGSGSGTDFTLTISSLQPEDFATYFCQQYSNYPLTFGGGTKVEIKR
VL1B
(SEQ ID NO: 12)
DIVMTQSPSSLSASVGDRVTITCKASQGVGSAIAWYQQKPGQSPKLLIYWASIRHTG
VPDRFTGSGSGTDFTLTISSLQPEDFATYFCQQYSNYPLTFGGGTKVEIKR
VL1C
(SEQ ID NO: 13)
DIVMTQSPSSVSASVGDRVTITCKASQGVGSAIAWYQQKPGQSPKLLIYWASIRHTG
VPDRFTGSGSGTDFTLTISSLQPEDFATYFCQQYSNYPLTFGGGTKLTVKR
VL1D
(SEQ ID NO: 14)

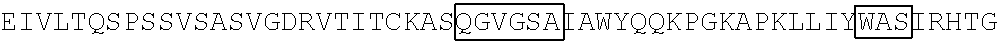

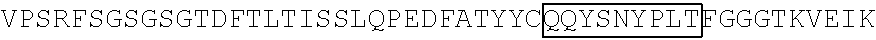

VL1E
(SEQ ID NO: 15)

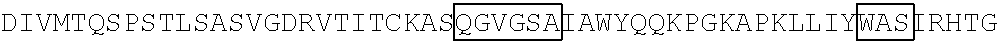

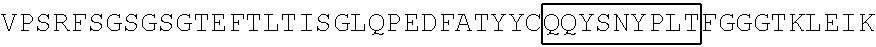

VL1F
(SEQ ID NO: 16)

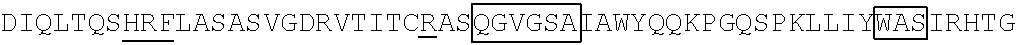

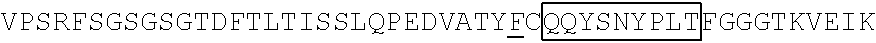

VL1G
(SEQ ID NO: 17)

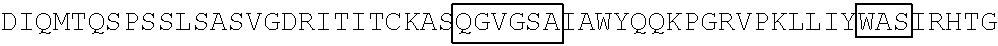

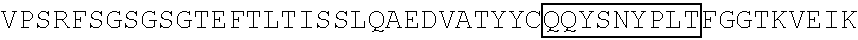

VL1H
(SEQ ID NO: 18)

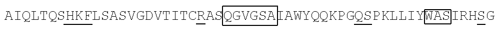

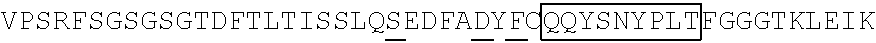

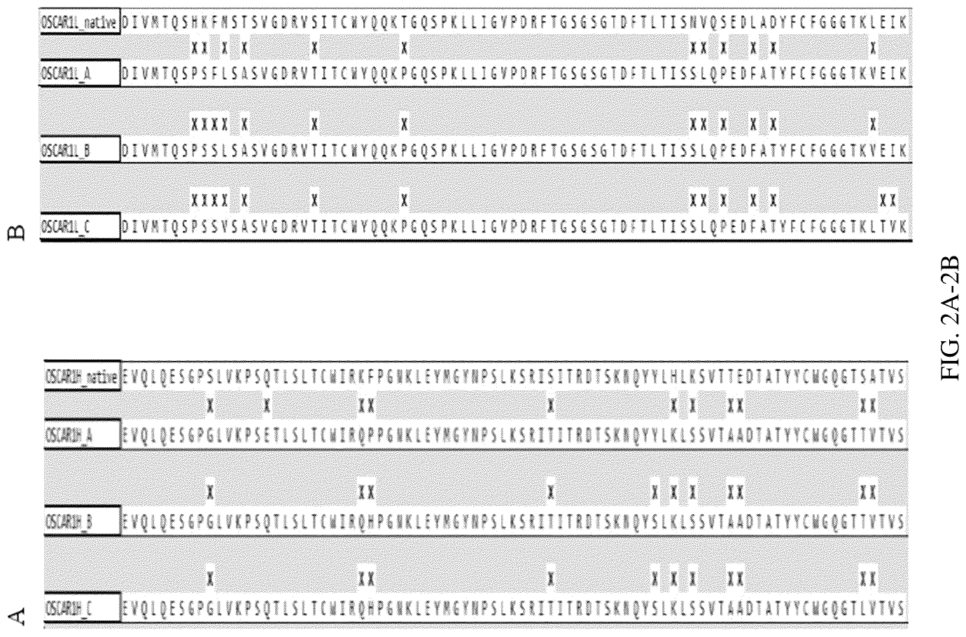

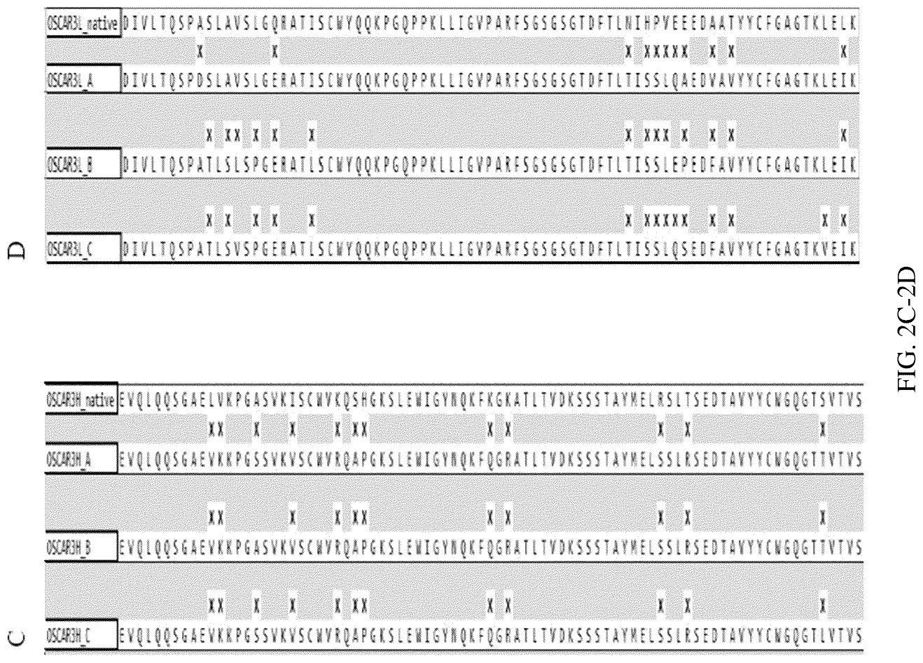

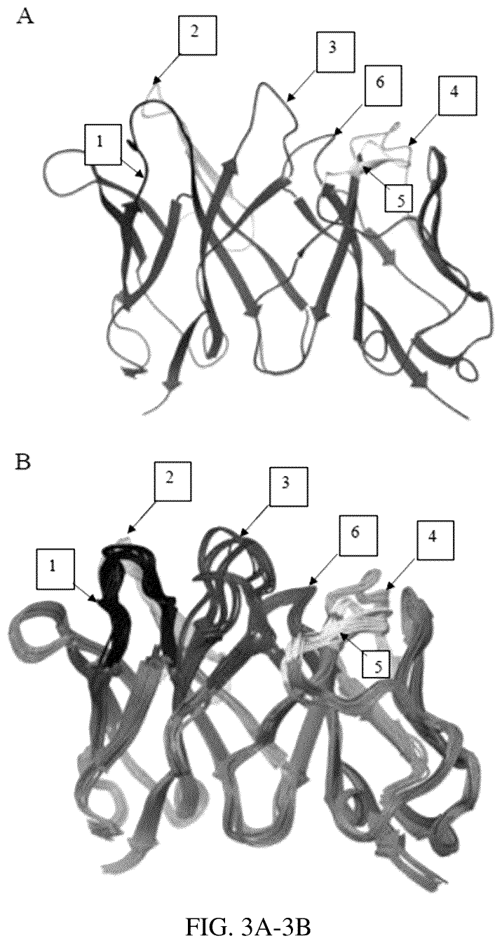

VL1A comprises substitutions in 12 positions relative to the native human framework sequence as depicted in FIG. 2B.

VL1B comprises substitutions in 13 positions relative to the native human framework sequence as depicted in FIG. 2B.

VL1C comprises substitutions in 14 positions relative to the native human framework sequence as depicted in FIG. 2B.

VL1F comprises substitutions in position 8, 9, 10, 24, 42, 43 and 87 (underlined above).

VL1H comprises substitutions in position 8, 9, 10, 24, 42, 43, 56, 80, 85 and 87 (underlined above).

We provide eight examples of TP1 VH sequences comprising human framework sequences:

VH's TP1:

VH1A
(SEQ ID NO: 19)
EVQLQESGPGLVKPSETLSLTCSVTGDSITSGYWNWIRQPPGNKLEYMGYISYSDTT
YYNPSLKSRITITRDTSKNQYYLKLSSVTAADTATYYCASAYYGSSLSMGNWGQGTT
VTVSS
VH1B
(SEQ ID NO: 20)
EVQLQESGPGLVKPSQTLSLTCSVTGDSITSGYWNWIRQHPGNKLEYMGYISYSDTT
YYNPSLKSRITITRDTSKNQYSLKLSSVTAADTATYYCASAYYGSSLSMGNWGQGTT
VTVSS
VH1C (SEQ ID NO: 21)
EVQLQESGPGLVKPSQTLSLTCSVTGDSITSGYWNWIRQHPGNKLEYMGYISYSDTT
YYNPSLKSRITITRDTSKNQYSLKLSSVTAADTATYYCASAYYGSSLSMGNWGQGTL
VTVSS
VH1D
(SEQ ID NO: 22)

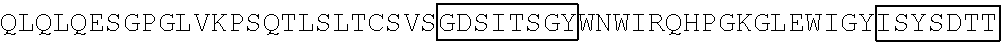

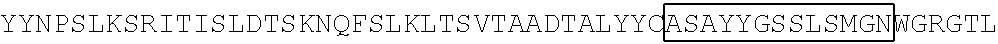

VTVSS
VH1E
(SEQ ID NO: 23)

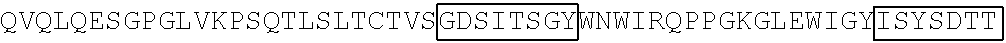

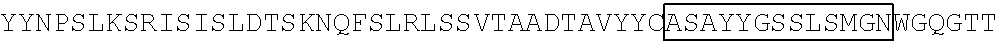

VTVSS
VH1F
(SEQ ID NO: 24)

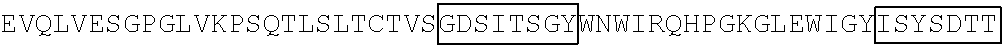

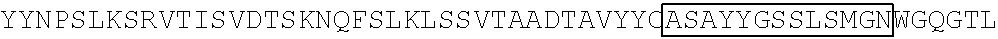

VTVSS
VH1G
(SEQ ID NO: 25)

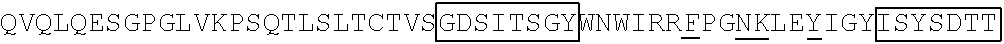

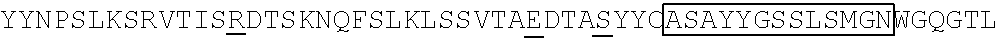

VTVSS
VH1H
(SEQ ID NO: 26)

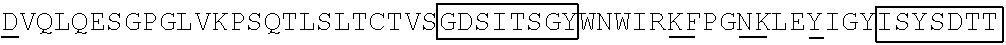

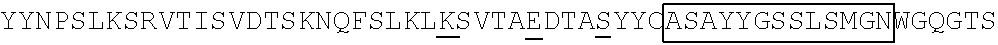

ATVSS

VH1A comprises substitutions in 11 positions relative to the native human framework sequence as depicted in FIG. 2A.

VH1B comprises substitutions in substitutions in 11 positions relative to the native human framework sequence as depicted in FIG. 2A.

VH1C comprises substitutions in substitutions in 11 positions relative to the native human framework sequence as depicted in FIG. 2A.

VH1G comprises substitutions in position 40, 43, 44, 47, 71, 84 and 89 (underlined above).

VH1H comprises substitutions in position 1, 39, 40, 43, 44, 47, 82, 84 and 89 (underlined above).

We provide eight examples of TP3 VL sequences comprising human framework sequences:

VL's TP3:

VL3A
(SEQ ID NO: 27)
DIVLTQSPDSLAVSLGERATISCRASKSVSTGYSYLHWYQQKPGQPPKLLIYLASNL
ESGVPARFSGSGSGTDFTLTISSLQAEDVAVYYCQHSRELPLTFGAGTKLEIKR
VL3B
(SEQ ID NO: 28)
DIVLTQSPATLSLSPGERATLSCRASKSVSTGYSYLHWYQQKPGQPPKLLIYLASNL
ESGVPARFSGSGSGTDFTLTISSLEPEDFAVYYCQHSRELPLTFGAGTKLEIKR
VL3C
(SEQ ID NO: 29)
DIVLTQSPATLSVSPGERATLSCRASKSVSTGYSYLHWYQQKPGQPPKLLIYLASNL
ESGVPARFSGSGSGTDFTLTISSLQSEDFAVYYCQHSRELPLTFGAGTKVEIKR
VL3D
(SEQ ID NO: 30)

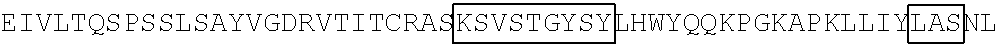

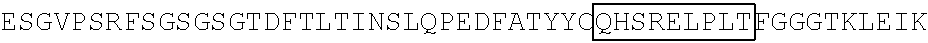

VL3E
(SEQ ID NO: 31)

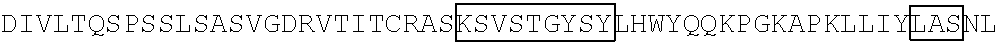

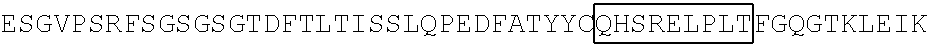

VL3F
(SEQ ID NO: 32)

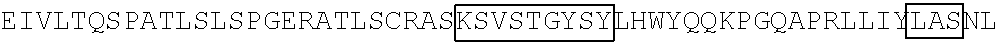

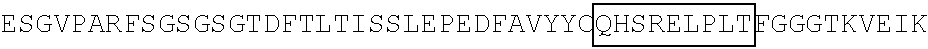

VL3G
(SEQ ID NO: 33)

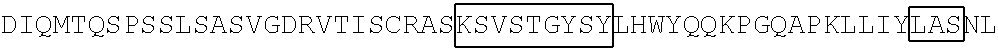

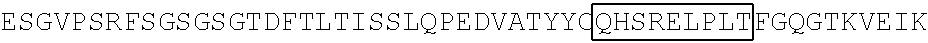

VL3H
(SEQ ID NO: 34)

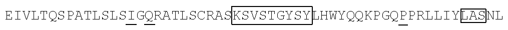

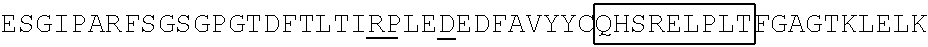

VL3A comprises substitutions in 11 positions relative to the native human framework sequence as depicted in FIG. 2D.

VL3B comprises substitutions in 14 positions relative to the native human framework sequence as depicted in FIG. 2D.

VL3C comprises substitutions in 15 positions relative to the native human framework sequence as depicted in FIG. 2D.

VL3H comprises substitutions in position 15, 17, 43, 76, 77, 80 (underlined above).

VH's TP3:

We provide eight examples of VH TP3 sequences comprising human framework sequences:

VH3A
(SEQ ID NO: 35)
EVQLQQSGAEVKKPGSSVKVSCKASGYTFTDYNMDWVRQAPGKSLEWIGDINPNYDS
TRYNQKFQGRATLTVDKSSSTAYMELSSLRSEDTAVYYCARGDYYVSSYGHDYAMDY
WGQGTTVTVSS
VH3B
(SEQ ID NO: 36)
EVQLQQSGAEVKKPGASVKVSCKASGYTFTDYNMDWVRQAPGKSLEWIGDINPNYDS
TRYNQKFQGRATLTVDKSSSTAYMELSSLRSEDTAVYYCARGDYYVSSYGHDYAMDY
WGQGTTVTVSS
VH3C
(SEQ ID NO: 37)
EVQLQQSGAEVKKPGSSVKVSCKASGYTFTDYNMDWVRQAPGKSLEWIGDINPNYDS
TRYNQKFQGRATLTVDKSSSTAYMELSSLRSEDTAVYYCARGDYYVSSYGHDYAMDY
WGQGTLVTVSS
VH3D
(SEQ ID NO: 38)

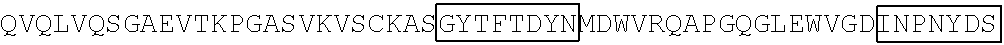

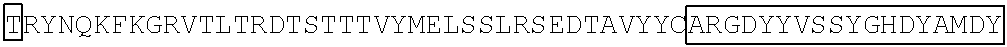

WGQGTTVTVSS
VH3E
(SEQ ID NO: 39)

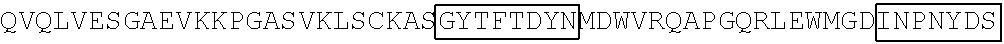

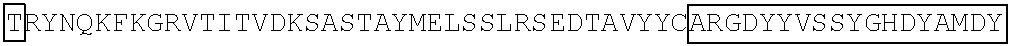

WGPGTTVTVSS
VH3F
(SEQ ID NO: 40)

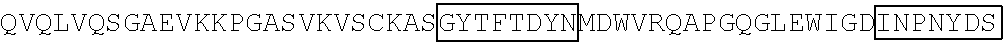

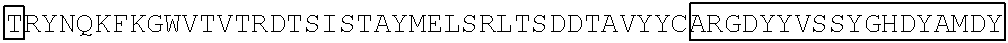

WGQGTMVTVSS
VH3G
(SEQ ID NO: 41)

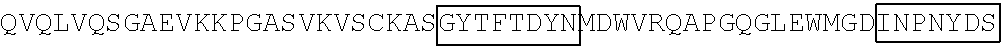

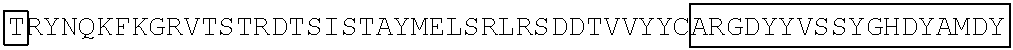

WGQGTSVTVSS
VH3H
(SEQ ID NO: 42)

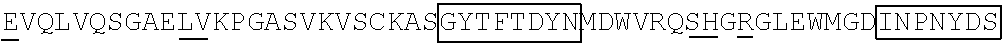

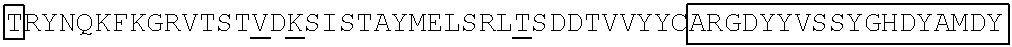

WGQGTSVTVSS

VH3A comprises substitutions in 12 positions relative to the native human framework sequence as depicted in FIG. 2C.

VH3B comprises substitutions in substitutions in 11 positions relative to the native human framework sequence as depicted in FIG. 2C.

VH3C comprises substitutions in substitutions in 12 positions relative to the native human framework sequence as depicted in FIG. 2C.

VH3H comprises substitutions in position 1, 11, 12, 40, 41, 43, 71, 73 and 83 (underlined above).

In antigen binding units, the human framework sequences may tolerate some variation without destroying the specificity and affinity to the target antigen. For example, substitutions of amino acid residues may be tolerated better than deletions or additions of amino acid residues.

However, the CDRs are generally more sensitive for variations, but occasionally conservative substitutions may be introduced without destroying the specificity and affinity.

The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid residue in the same family.

Examples of scFv's provided herein:

| Name | Sequence |
|---|---|
| VL1A- | DIVMTQSPSFLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1A | EIKRGGGGSGGGGSGGGGSGGGGSEVQLQESGPGLVKPSETLSLTCSVTGDS |
| (SEQ ID NO: 43) | ITSGYWNWIRQPPGNKLEYMGYISYSDTTYYNPSLKSRITITRDTSKNQYYL |
| | KLSSVTAADTATYYCASAYYGSSLSMGNWGQGTTVTVSS |
| VL1A- | DIVMTQSPSFLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1B | EIKRGGGGSGGGGSGGGGSGGGGSEVQLQESGPGLVKPSQTLSLTCSVTGDS |
| (SEQ ID NO: 44) | ITSGYWNWIRQHPGNKLEYMGYISYSDTTYYNPSLKSRITITRDTSKNQYSL |
| | KLSSVTAADTATYYCASAYYGSSLSMGNWGQGTTVTVSS |
| VL1A- | DIVMTQSPSFLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1C | EIKRGGGGSGGGGSGGGGSGGGGSEVQLQESGPGLVKPSQTLSLTCSVTGDS |
| (SEQ ID NO: 45) | ITSGYWNWIRQHPGNKLEYMGYISYSDTTYYNPSLKSRITITRDTSKNQYSL |
| | KLSSVTAADTATYYCASAYYGSSLSMGNWGQGTLVTVSS |
| VL1B- | DIVMTQSPSSLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1A | EIKRGGGGSGGGGSGGGGSGGGGSDIVMTQSPSSLSASVGDRVTITCKASQG |
| (SEQ ID NO: 46) | VGSAIAWYQQKPGQSPKLLIYWASIRHTGVPDRFTGSGSGTDFTLTISSLQP |
| | EDFATYFCQQYSNYPLTFGGGTKVEIKR |
| VL1B- | DIVMTQSPSSLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1B | EIKRGGGGSGGGGSGGGGSGGGGSEVQLQESGPGLVKPSQTLSLTCSVTGDS |
| (SEQ ID NO: 47) | ITSGYWNWIRQHPGNKLEYMGYISYSDTTYYNPSLKSRITITRDTSKNQYSL |
| | KLSSVTAADTATYYCASAYYGSSLSMGNWGQGTTVTVSS |
| VL1B- | DIVMTQSPSSLSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKV |
| VH1C | EIKRGGGGSGGGGSGGGGSGGGGSEVQLQESGPGLVKPSQTLSLTCSVTGDS |
| (SEQ ID NO: 48) | ITSGYWNWIRQHPGNKLEYMGYISYSDTTYYNPSLKSRITITRDTSKNQYSL |
| | KLSSVTAADTATYYCASAYYGSSLSMGNWGQGTLVTVSS |
| VL1C- | DIVMTQSPSSVSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKL |
| VH1A | TVKRGGGGSGGGGSGGGGSGGGGSEVQLQESGPGLVKPSETLSLTCSVTGDS |
| (SEQ ID NO: 49) | ITSGYWNWIRQPPGNKLEYMGYISYSDTTYYNPSLKSRITITRDTSKNQYYL |
| | KLSSVTAADTATYYCASAYYGSSLSMGNWGQGTTVTVSS |
| VL1C- | DIVMTOSPSSVSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKL |
| VH1B | TVKRGGGGSGGGGSGGGGSGGGGSEVQLQESGPGLVKPSQTLSLTCSVTGDS |
| (SEQ ID NO: 50) | ITSGYWNWIRQHPGNKLEYMGYISYSDTTYYNPSLKSRITITRDTSKNQYSL |
| | KLSSVTAADTATYYCASAYYGSSLSMGNWGQGTTVTVSS |
| VL1C- | DIVMTQSPSSVSASVGDRVTITCKASQGVGSAIAWYQQKPGQSPKLLIYWAS |
| linker- | IRHTGVPDRFTGSGSGTDFTLTISSLQPEDFATYFCQQYSNYPLTFGGGTKL |
| VH1C | TVKRGGGGSGGGGSGGGGSGGGGSEVQLQESGPGLVKPSQTLSLTCSVTGDS |
| (SEQ ID NO: 51) | ITSGYWNWIRQHPGNKLEYMGYISYSDTTYYNPSLKSRITITRDTSKNQYSL |
| | KLSSVTAADTATYYCASAYYGSSLSMGNWGQGTLVTVSS |
| VL1D- | EIVLTQSPSSVSASVGDRVTITCKASQGVGSAIAWYQQKPGKAPKLLIYWAS |
| linker- | IRHTGVPSRFSGSGSGTDFTLTISSLQPEDFATYYCQQYSNYPLTFGGGTKV |
| VH1D | EIKRGGGGSGGGGSGGGGSGGGGSQLQLQESGPGLVKPSQTLSLTCSVSGDS |
| (SEQ ID NO: 52) | ITSGYWNWIRQHPGKGLEWIGYISYSDTTYYNPSLKSRITISLDTSKNQFSL |
| | KLTSVTAADTALYYCASAYYGSSLSMGNWGRGTLVTVSSD |
| VL1E- | DIVMTQSPSTLSASVGDRVTITCKASQGVGSAIAWYQQKPGKAPKLLIYWAS |
| linker- | IRHTGVPSRFSGSGSGTEFTLTISGLQPEDFATYYCQQYSNYPLTFGGGTKL |
| VH1D | EIKRGGGGSGGGGSGGGGSGGGGSQLQLQESGPGLVKPSQTLSLTCSVSGDS |
| (SEQ ID NO: 53) | ITSGYWNWIRQHPGKGLEWIGYISYSDTTYYNPSLKSRITISLDTSKNQFSL |
| | KLTSVTAADTALYYCASAYYGSSLSMGNWGRGTLVTVSSD |
| VL1F- | DIQLTQSHRFLSASVGDRVTITCRASQGVGSAIAWYQQKPGQSPKLLIYWAS |
| linker- | IRHTGVPSRFSGSGSGTDFTLTISSLQPEDVATYFCQQYSNYPLTFGGGTKV |
| VH1D | EIKRGGGGSGGGGSGGGGSGGGGSQLQLQESGPGLVKPSQTLSLTCSVSGDS |
| (SEQ ID NO: 54) | ITSGYWNWIRQHPGKGLEWIGYISYSDTTYYNPSLKSRITISLDTSKNQFSL |
| | KLTSVTAADTALYYCASAYYGSSLSMGNWGRGTLVTVSSD |
| VL1G- | DIQMTQSPSSLSASVGDRITITCKASQGVGSAIAWYQQKPGRVPKLLIYWAS |
| linker- | IRHTGVPSRFSGSGSGTEFTLTISSLQAEDVATYYCQQYSNYPLTFGGGTKV |
| VH1D | EIKRGGGGSGGGGSGGGGSGGGGSQLQLQESGPGLVKPSQTLSLTCSVSGDS |
| (SEQ ID NO: 55) | ITSGYWNWIRQHPGKGLEWIGYISYSDTTYYNPSLKSRITISLDTSKNQFSL |
| | KLTSVTAADTALYYCASAYYGSSLSMGNWGRGTLVTVSSD |
| VL1H- | AIQLTQSHKFLSASVGDRVTITCRASQGVGSAIAWYQQKPGQSPKLLIYWAS |
| linker- | IRHSGVPSRFSGSGSGTDFTLTISSLQSEDFADYFCQQYSNYPLTFGGGTKL |
| VH1D | EIKRGGGGSGGGGSGGGGSGGGGSQLQLQESGPGLVKPSQTLSLTCSVSGDS |
| (SEQ ID NO: 56) | ITSGYWNWIRQHPGKGLEWIGYISYSDTTYYNPSLKSRITISLDTSKNQFSL |
| | KLTSVTAADTALYYCASAYYGSSLSMGNWGRGTLVTVSSD |
| VL1D- | EIVLTQSPSSVSASVGDRVTITCKASQGVGSAIAWYQQKPGKAPKLLIYWAS |
| linker- | IRHTGVPSRFSGSGSGTDFTLTISSLQPEDFATYYCQQYSNYPLTFGGGTKV |
| VH1E | EIKRGGGGSGGGGSGGGGSGGGGSQVQLQESGPGLVKPSQTLSLTCTVSGDS |
| (SEQ ID NO: 57) | ITSGYWNWIRQPPGKGLEWIGYISYSDTTYYNPSLKSRISISLDTSKNQFSL |
| | RLSSVTAADTAVYYCASAYYGSSLSMGNWGQGTTVTVSSD |
| VL1E- | DIVMTOSPSTLSASVGDRVTITCKASQGVGSAIAWYQQKPGKAPKLLIYWAS |
| linker- | IRHTGVPSRFSGSGSGTEFTLTISGLQPEDFATYYCQQYSNYPLTFGGGTKL |
| VH1E | EIKRGGGGSGGGGSGGGGSGGGGSQVQLQESGPGLVKPSQTLSLTCTVSGDS |
| (SEQ ID NO: 58) | ITSGYWNWIRQPPGKGLEWIGYISYSDTTYYNPSLKSRISISLDTSKNQFSL |
| | RLSSVTAADTAVYYCASAYYGSSLSMGNWGQGTTVTVSSD |
| VL1F- | DIQLTQSHRFLSASVGDRVTITCRASQGVGSAIAWYQQKPGQSPKLLIYWAS |
| linker- | IRHTGVPSRFSGSGSGTDFTLTISSLQPEDVATYFCQQYSNYPLTFGGGTKV |
| VH1E | EIKRGGGGSGGGGSGGGGSGGGGSQVQLQESGPGLVKPSQTLSLTCTVSGDS |
| (SEQ ID NO: 59) | ITSGYWNWIRQPPGKGLEWIGYISYSDTTYYNPSLKSRISISLDTSKNQFSL |
| | RLSSVTAADTAVYYCASAYYGSSLSMGNWGQGTTVTVSSD |
| VL1G- | DIQMTQSPSSLSASVGDRITITCKASQGVGSAIAWYQQKPGRVPKLLIYWAS |
| linker- | IRHTGVPSRFSGSGSGTEFTLTISSLQAEDVATYYCQQYSNYPLTFGGGTKV |
| VH1E | EIKRGGGGSGGGGSGGGGSGGGGSQVQLQESGPGLVKPSQTLSLTCTVSGDS |
| (SEQ ID NO: 60) | ITSGYWNWIRQPPGKGLEWIGYISYSDTTYYNPSLKSRISISLDTSKNQFSL |
| | RLSSVTAADTAVYYCASAYYGSSLSMGNWGQGTTVTVSSD |
| VL1H- | AIQLTQSHKFLSASVGDRVTITCRASQGVGSAIAWYQQKPGQSPKLLIYWAS |
| linker- | IRHSGVPSRFSGSGSGTDFTLTISSLQSEDFADYFCQQYSNYPLTFGGGTKL |
| VH1E | EIKRGGGGSGGGGSGGGGSGGGGSQVQLQESGPGLVKPSQTLSLTCTVSGDS |
| (SEQ ID NO: 61) | ITSGYWNWIRQPPGKGLEWIGYISYSDTTYYNPSLKSRISISLDTSKNQFSL |
| | RLSSVTAADTAVYYCASAYYGSSLSMGNWGQGTTVTVSSD |
| VL1D- | EIVLTQSPSSVSASVGDRVTITCKASQGVGSAIAWYQQKPGKAPKLLIYWAS |
| linker- | IRHTGVPSRFSGSGSGTDFTLTISSLQPEDFATYYCQQYSNYPLTFGGGTKV |
| VH1F | EIKRGGGGSGGGGSGGGGSGGGGSEVQLVESGPGLVKPSQTLSLTCTVSGDS |
| (SEQ ID NO: 62) | ITSGYWNWIRQHPGKGLEWIGYISYSDTTYYNPSLKSRVTISVDTSKNQFSL |
| | KLSSVTAADTAVYYCASAYYGSSLSMGNWGQGTLVTVSSD |
| VL1E- | DIVMTQSPSTLSASVGDRVTITCKASQGVGSAIAWYQQKPGKAPKLLIYWAS |
| linker- | IRHTGVPSRFSGSGSGTEFTLTISGLQPEDFATYYCQQYSNYPLTFGGGTKL |
| VH1F | EIKRGGGGSGGGGSGGGGSGGGGSEVQLVESGPGLVKPSQTLSLTCTVSGDS |
| (SEQ ID NO: 63) | ITSGYWNWIRQHPGKGLEWIGYISYSDTTYYNPSLKSRVTISVDTSKNQFSL |
| | KLSSVTAADTAVYYCASAYYGSSLSMGNWGQGTLVTVSSD |
| VL1F- | DIQLTQSHRFLSASVGDRVTITCRASQGVGSAIAWYQQKPGQSPKLLIYWAS |
| linker- | IRHTGVPSRFSGSGSGTDFTLTISSLQPEDVATYFCQQYSNYPLTFGGGTKV |
| VH1F | EIKRGGGGSGGGGSGGGGSGGGGSEVQLVESGPGLVKPSQTLSLTCTVSGDS |
| (SEQ ID NO: 64) | ITSGYWNWIRQHPGKGLEWIGYISYSDTTYYNPSLKSRVTISVDTSKNQFSL |
| | KLSSVTAADTAVYYCASAYYGSSLSMGNWGQGTLVTVSSD |
| VL1G- | DIQMTQSPSSLSASVGDRITITCKASQGVGSAIAWYQQKPGRVPKLLIYWAS |
| linker- | IRHTGVPSRFSGSGSGTEFTLTISSLQAEDVATYYCQQYSNYPLTFGGGTKV |
| VH1F | EIKRGGGGSGGGGSGGGGSGGGGSEVQLVESGPGLVKPSQTLSLTCTVSGDS |
| (SEQ ID NO: 65) | ITSGYWNWIRQHPGKGLEWIGYISYSDTTYYNPSLKSRVTISVDTSKNQFSL |
| | KLSSVTAADTAVYYCASAYYGSSLSMGNWGQGTLVTVSSD |
| VL1H- | AIQLTQSHKFLSASVGDRVTITCRASQGVGSAIAWYQQKPGQSPKLLIYWAS |
| linker- | IRHSGVPSRFSGSGSGTDFTLTISSLQSEDFADYFCQQYSNYPLTFGGGTKL |
| VH1F | EIKRGGGGSGGGGSGGGGSGGGGSEVQLVESGPGLVKPSQTLSLTCTVSGDS |
| (SEQ ID NO: 66) | ITSGYWNWIRQHPGKGLEWIGYISYSDTTYYNPSLKSRVTISVDTSKNQFSL |
| | KLSSVTAADTAVYYCASAYYGSSLSMGNWGQGTLVTVSSD |
| VL1D- | EIVLTQSPSSVSASVGDRVTITCKASQGVGSAIAWYQQKPGKAPKLLIYWAS |
| linker- | IRHTGVPSRFSGSGSGTDFTLTISSLOPEDFATYYCQQYSNYPLTFGGGTKV |
| VH1G | EIKRGGGGSGGGGSGGGGSGGGGSQVQLQESGPGLVKPSQTLSLTCTVSGDS |
| (SEQ ID NO: 67) | ITSGYWNWIRRFPGNKLEYIGYISYSDTTYYNPSLKSRVTISRDTSKNQFSL |
| | KLSSVTAEDTASYYCASAYYGSSLSMGNWGQGTLVTVSSD |
| VL1E- | DIVMTQSPSTLSASVGDRVTITCKASQGVGSAIAWYQQKPGKAPKLLIYWAS |
| linker- | IRHTGVPSRFSGSGSGTEFTLTISGLQPEDFATYYCQQYSNYPLTFGGGTKL |
| VH1G | EIKRGGGGSGGGGSGGGGSGGGGSQVQLQESGPGLVKPSQTLSLTCTVSGDS |
| (SEQ ID NO: 68) | ITSGYWNWIRRFPGNKLEYIGYISYSDTTYYNPSLKSRVTISRDTSKNQFSL |
| | KLSSVTAEDTASYYCASAYYGSSLSMGNWGQGTLVTVSSD |
| VL1F- | DIQLTQSHRFLSASVGDRVTITCRASQGVGSAIAWYQQKPGQSPKLLIYWAS |
| linker- | IRHTGVPSRFSGSGSGTDFTLTISSLQPEDVATYFCQQYSNYPLTFGGGTKV |
| VH1G | EIKRGGGGSGGGGSGGGGSGGGGSQVQLQESGPGLVKPSQTLSLTCTVSGDS |
| (SEQ ID NO: 69) | ITSGYWNWIRRFPGNKLEYIGYISYSDTTYYNPSLKSRVTISRDTSKNQFSL |
| | KLSSVTAEDTASYYCASAYYGSSLSMGNWGQGTLVTVSSD |
| VL1G- | DIQMTQSPSSLSASVGDRITITCKASQGVGSAIAWYQQKPGRVPKLLIYWAS |
| linker- | IRHTGVPSRFSGSGSGTEFTLTISSLQAEDVATYYCQQYSNYPLTFGGGTKV |
| VH1G | EIKRGGGGSGGGGSGGGGSGGGGSQVQLQESGPGLVKPSQTLSLTCTVSGDS |
| (SEQ ID NO: 70) | ITSGYWNWIRRFPGNKLEYIGYISYSDTTYYNPSLKSRVTISRDTSKNQFSL |
| | KLSSVTAEDTASYYCASAYYGSSLSMGNWGQGTLVTVSSD |
| VL1H- | AIQLTQSHKFLSASVGDRVTITCRASQGVGSAIAWYQQKPGQSPKLLIYWAS |
| linker- | IRHSGVPSRFSGSGSGTDFTLTISSLQSEDFADYFCQQYSNYPLTFGGGTKL |
| VH1G | EIKRGGGGSGGGGSGGGGSGGGGSQVQLQESGPGLVKPSQTLSLTCTVSGDS |
| (SEQ ID NO: 71) | ITSGYWNWIRRFPGNKLEYIGYISYSDTTYYNPSLKSRVTISRDTSKNQFSL |
| | KLSSVTAEDTASYYCASAYYGSSLSMGNWGQGTLVTVSSD |
| VL1D- | EIVLTQSPSSVSASVGDRVTITCKASQGVGSAIAWYQQKPGKAPKLLIYWAS |
| linker- | IRHTGVPSRFSGSGSGTDFTLTISSLQPEDFATYYCQQYSNYPLTFGGGTKV |
| VH1H | EIKRGGGGSGGGGSGGGGSGGGGSDVQLQESGPGLVKPSQTLSLTCTVSGDS |
| (SEQ ID NO: 72) | ITSGYWNWIRKFPGNKLEYIGYISYSDTTYYNPSLKSRVTISVDTSKNQFSL |
| | KLKSVTAEDTASYYCASAYYGSSLSMGNWGQGTSATVSSD |
| VL1E- | DIVMTQSPSTLSASVGDRVTITCKASQGVGSAIAWYQQKPGKAPKLLIYWAS |
| linker- | IRHTGVPSRFSGSGSGTEFTLTISGLQPEDFATYYCQQYSNYPLTFGGGTKL |
| VH1H | EIKRGGGGSGGGGSGGGGSGGGGSDVQLQESGPGLVKPSQTLSLTCTVSGDS |
| (SEQ ID NO: 73) | ITSGYWNWIRKFPGNKLEYIGYISYSDTTYYNPSLKSRVTISVDTSKNQFSL |
| | KLKSVTAEDTASYYCASAYYGSSLSMGNWGQGTSATVSSD |
| VL1F- | DIQLTQSHRFLSASVGDRVTITCRASQGVGSAIAWYQQKPGQSPKLLIYWAS |
| linker- | IRHTGVPSRFSGSGSGTDFTLTISSLQPEDVATYFCQQYSNYPLTFGGGTKV |
| VH1H | EIKRGGGGSGGGGSGGGGSGGGGSDVQLQESGPGLVKPSQTLSLTCTVSGDS |
| (SEQ ID NO: 74) | ITSGYWNWIRKFPGNKLEYIGYISYSDTTYYNPSLKSRVTISVDTSKNQFSL |
| | KLKSVTAEDTASYYCASAYYGSSLSMGNWGQGTSATVSSD |
| VL1G- | DIQMTQSPSSLSASVGDRITITCKASQGVGSAIAWYQQKPGRVPKLLIYWAS |
| linker- | IRHTGVPSRFSGSGSGTEFTLTISSLQAEDVATYYCQQYSNYPLTFGGGTKV |
| VH1H | EIKRGGGGSGGGGSGGGGSGGGGSDVQLQESGPGLVKPSQTLSLTCTVSGDS |
| (SEQ ID NO: 75) | ITSGYWNWIRKFPGNKLEYIGYISYSDTTYYNPSLKSRVTISVDTSKNQFSL |
| | KLKSVTAEDTASYYCASAYYGSSLSMGNWGQGTSATVSSD |
| VL1H- | AIQLTQSHKFLSASVGDRVTITCRASQGVGSAIAWYQQKPGQSPKLLIYWAS |
| linker- | IRHSGVPSRFSGSGSGTDFTLTISSLQSEDFADYFCQQYSNYPLTFGGGTKL |
| VH1H | EIKRGGGGSGGGGSGGGGSGGGGSDVQLQESGPGLVKPSQTLSLTCTVSGDS |
| (SEQ ID NO: 76) | ITSGYWNWIRKFPGNKLEYIGYISYSDTTYYNPSLKSRVTISVDTSKNQFSL |
| | KLKSVTAEDTASYYCASAYYGSSLSMGNWGQGTSATVSSD |

In one particular embodiment, the scFv may be VL1E-linker-VH1G, VH1G-linker-VL1E, VL1D-linker-VH1G or VH1G-linker-VL1D.

Further examples of scFv's provided herein:

| Name | Sequence |
|---|---|
| VL3A- | DIVLTQSPDSLAVSLGERATISCRASKSVSTGYSYLHWYQQKPGQPPKLLIY |
| linker- | LASNLESGVPARFSGSGSGTDFTLTISSLQAEDVAVYYCQHSRELPLTFGAG |
| VH3A | TKLEIKRGGGGSGGGGSGGGGSGGGGSEVQLQQSGAEVKKPGSSVKVSCKAS |
| (SEQ ID NO: 77) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3A- | DIVLTQSPDSLAVSLGERATISCRASKSVSTGYSYLHWYQQKPGQPPKLLIY |
| linker- | LASNLESGVPARFSGSGSGTDFTLTISSLQAEDVAVYYCQHSRELPLTFGAG |
| VH3B | TKLEIKRGGGGSGGGGSGGGGSGGGGSEVQLQQSGAEVKKPGASVKVSCKAS |
| (SEQ ID NO: 78) | GYTFTDYNMDWVROAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3A- | DIVLTQSPDSLAVSLGERATISCRASKSVSTGYSYLHWYQQKPGQPPKLLIY |
| linker- | LASNLESGVPARFSGSGSGTDFTLTISSLQAEDVAVYYCQHSRELPLTFGAG |
| VH3C | TKLEIKRGGGGSGGGGSGGGGSGGGGSEVQLQQSGAEVKKPGSSVKVSCKAS |
| (SEQ ID NO: 79) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTLVTVSS |
| VL3B- | DIVLTQSPATLSLSPGERATLSCRASKSVSTGYSYLHWYQQKPGQPPKLLIY |
| linker- | LASNLESGVPARFSGSGSGTDFTLTISSLEPEDFAVYYCQHSRELPLTFGAG |
| VH3A | TKLEIKRGGGGSGGGGSGGGGSGGGGSEVQLQQSGAEVKKPGSSVKVSCKAS |
| (SEQ ID NO: 80) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3B- | DIVLTQSPATLSLSPGERATLSCRASKSVSTGYSYLHWYQQKPGQPPKLLIY |
| linker- | LASNLESGVPARFSGSGSGTDFTLTISSLEPEDFAVYYCQHSRELPLTFGAG |
| VH3B | TKLEIKRGGGGSGGGGSGGGGSGGGGSEVQLQQSGAEVKKPGASVKVSCKAS |
| (SEQ ID NO: 81) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3B- | DIVLTQSPATLSLSPGERATLSCRASKSVSTGYSYLHWYQQKPGQPPKLLIY |
| linker- | LASNLESGVPARFSGSGSGTDFTLTISSLEPEDFAVYYCQHSRELPLTFGAG |
| VH3C | TKLEIKRGGGGSGGGGSGGGGSGGGGSEVQLQQSGAEVKKPGSSVKVSCKAS |
| (SEQ ID NO: 82) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTLVTVSS |
| VL3C- | DIVLTQSPATLSVSPGERATLSCRASKSVSTGYSYLHWYQQKPGQPPKLLIY |
| linker- | LASNLESGVPARFSGSGSGTDFTLTISSLQSEDFAVYYCQHSRELPLTFGAG |
| VH3A | TKVEIKRGGGGSGGGGSGGGGSGGGGSEVQLQQSGAEVKKPGSSVKVSCKAS |
| (SEQ ID NO: 83) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3C- | DIVLTQSPATLSVSPGERATLSCRASKSVSTGYSYLHWYQQKPGQPPKLLIY |
| linker- | LASNLESGVPARFSGSGSGTDFTLTISSLQSEDFAVYYCQHSRELPLTFGAG |
| VH3B | TKVEIKRGGGGSGGGGSGGGGSGGGGSEVQLQQSGAEVKKPGASVKVSCKAS |
| (SEQ ID NO: 84) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTTVTVSS |
| VL3C- | DIVLTQSPATLSVSPGERATLSCRASKSVSTGYSYLHWYQQKPGQPPKLLIY |
| linker- | LASNLESGVPARFSGSGSGTDFTLTISSLQSEDFAVYYCQHSRELPLTFGAG |
| VHC | TKVEIKRGGGGSGGGGSGGGGSGGGGSEVQLQQSGAEVKKPGSSVKVSCKAS |
| (SEQ ID NO: 85) | GYTFTDYNMDWVRQAPGKSLEWIGDINPNYDSTRYNQKFQGRATLTVDKSSS |
| | TAYMELSSLRSEDTAVYYCARGDYYVSSYGHDYAMDYWGQGTLVTVSS |
| VL3D- | EIVLTQSPSSLSAYVGDRVTITCRASKSVSTGYSYLHWYQQKPGKAPKLLIY |
| linker- | LASNLESGVPSRFSGSGSGTDFTLTINSLQPEDFATYYCQHSRELPLTFGGG |
| VH3D | TKLEIKRGGGGSGGGGSGGGGSGGGGSQVQLVQSGAEVTKPGASVKVSCKAS |
| (SEQ ID NO: 86) | GYTFTDYNMDWVRQAPGQGLEWVGDINPNYDSTRYNQKFKGRVTLTRDTSTT |
| | TVYMELSSLRSEDTAVYYCARGDYYVSSYGHDYAMDYWGQGTTVTVSSD |
| VL3E- | DIVLTQSPSSLSASVGDRVTITCRASKSVSTGYSYLHWYQQKPGKAPKLLIY |
| linker- | LASNLESGVPSRFSGSGSGTDFTLTISSLQPEDFATYYCQHSRELPLTFGQG |
| VH3D | TKLEIKRGGGGSGGGGSGGGGSGGGGSQVQLVQSGAEVTKPGASVKVSCKAS |
| (SEQ ID NO: 87) | GYTFTDYNMDWVRQAPGQGLEWVGDINPNYDSTRYNQKFKGRVTLTRDTSTT |
| | TVYMELSSLRSEDTAVYYCARGDYYVSSYGHDYAMDYWGQGTTVTVSSD |
| VL3F- | EIVLTQSPATLSLSPGERATLSCRASKSVSTGYSYLHWYQQKPGQAPRLLIY |
| linker- | LASNLESGVPARFSGSGSGTDFTLTISSLEPEDFAVYYCQHSRELPLTFGGG |
| VH3D | TKVEIKRGGGGSGGGGSGGGGSGGGGSQVQLVQSGAEVTKPGASVKVSCKAS |
| (SEQ ID NO: 88) | GYTFTDYNMDWVRQAPGQGLEWVGDINPNYDSTRYNQKFKGRVTLTRDTSTT |
| | TVYMELSSLRSEDTAVYYCARGDYYVSSYGHDYAMDYWGQGTTVTVSSD |
| VL3G- | DIQMTQSPSSLSASVGDRVTISCRASKSVSTGYSYLHWYQQKPGQAPKLLIY |
| linker- | LASNLESGVPSRFSGSGSGTDFTLTISSLQPEDVATYYCQHSRELPLTFGQG |
| VH3D | TKVEIKRGGGGSGGGGSGGGGSGGGGSQVQLVQSGAEVTKPGASVKVSCKAS |
| (SEQ ID NO: 89) | GYTFTDYNMDWVRQAPGQGLEWVGDINPNYDSTRYNQKFKGRVTLTRDTSTT |
| | TVYMELSSLRSEDTAVYYCARGDYYVSSYGHDYAMDYWGQGTTVTVSSD |
| VL3H- | EIVLTQSPATLSLSIGQRATLSCRASKSVSTGYSYLHWYQQKPGQPPRLLIY |
| linker- | LASNLESGIPARFSGSGPGTDFTLTIRPLEDEDFAVYYCQHSRELPLTFGAG |
| VH3D | TKLELKRGGGGSGGGGSGGGGSGGGGSQVQLVQSGAEVTKPGASVKVSCKAS |
| (SEQ ID NO: 90) | GYTFTDYNMDWVRQAPGQGLEWVGDINPNYDSTRYNQKFKGRVTLTRDTSTT |
| | TVYMELSSLRSEDTAVYYCARGDYYVSSYGHDYAMDYWGQGTTVTVSSD |
| VL3D- | EIVLTQSPSSLSAYVGDRVTITCRASKSVSTGYSYLHWYQQKPGKAPKLLIY |
| linker- | LASNLESGVPSRFSGSGSGTDFTLTINSLQPEDFATYYCQHSRELPLTFGGG |
| VH3E | TKLEIKRGGGGSGGGGSGGGGSGGGGSQVQLVESGAEVKKPGASVKLSCKAS |
| (SEQ ID NO: 91) | GYTFTDYNMDWVRQAPGORLEWMGDINPNYDSTRYNQKFKGRVTITVDKSAS |
| | TAYMELSSLRSEDTAVYYCARGDYYVSSYGHDYAMDYWGPGTTVTVSSD |
| VL3E- | DIVLTQSPSSLSASVGDRVTITCRASKSVSTGYSYLHWYQQKPGKAPKLLIY |
| linker- | LASNLESGVPSRFSGSGSGTDFTLTISSLQPEDFATYYCQHSRELPLTFGQG |
| VH3E | TKLEIKRGGGGSGGGGSGGGGSGGGGSQVQLVESGAEVKKPGASVKLSCKAS |
| (SEQ ID NO: 92) | GYTFTDYNMDWVRQAPGORLEWMGDINPNYDSTRYNQKFKGRVTITVDKSAS |
| | TAYMELSSLRSEDTAVYYCARGDYYVSSYGHDYAMDYWGPGTTVTVSSD |
| VL3F- | EIVLTQSPATLSLSPGERATLSCRASKSVSTGYSYLHWYQQKPGQAPRLLIY |
| linker- | LASNLESGVPARFSGSGSGTDFTLTISSLEPEDFAVYYCQHSRELPLTFGGG |
| VH3E | TKVEIKRGGGGSGGGGSGGGGSGGGGSQVQLVESGAEVKKPGASVKLSCKAS |
| (SEQ ID NO: 93) | GYTFTDYNMDWVRQAPGORLEWMGDINPNYDSTRYNQKFKGRVTITVDKSAS |
| | TAYMELSSLRSEDTAVYYCARGDYYVSSYGHDYAMDYWGPGTTVTVSSD |
| VL3G- | DIQMTQSPSSLSASVGDRVTISCRASKSVSTGYSYLHWYQQKPGQAPKLLIY |
| linker- | LASNLESGVPSRFSGSGSGTDFTLTISSLQPEDVATYYCQHSRELPLTFGQG |
| VH3E | TKVEIKRGGGGSGGGGSGGGGSGGGGSQVQLVESGAEVKKPGASVKLSCKAS |
| (SEQ ID NO: 94) | GYTFTDYNMDWVRQAPGQRLEWMGDINPNYDSTRYNQKFKGRVTITVDKSAS |
| | TAYMELSSLRSEDTAVYYCARGDYYVSSYGHDYAMDYWGPGTTVTVSSD |
| VL3H- | EIVLTQSPATLSLSIGQRATLSCRASKSVSTGYSYLHWYQQKPGQPPRLLIY |
| linker- | LASNLESGIPARFSGSGPGTDFTLTIRPLEDEDFAVYYCQHSRELPLTFGAG |
| VH3E | TKLELKRGGGGSGGGGSGGGGSGGGGSQVQLVESGAEVKKPGASVKLSCKAS |
| (SEQ ID NO: 95) | GYTFTDYNMDWVRQAPGQRLEWMGDINPNYDSTRYNQKFKGRVTITVDKSAS |
| | TAYMELSSLRSEDTAVYYCARGDYYVSSYGHDYAMDYWGPGTTVTVSSD |
| VL3D- | EIVLTQSPSSLSAYVGDRVTITCRASKSVSTGYSYLHWYQQKPGKAPKLLIY |
| linker- | LASNLESGVPSRFSGSGSGTDFTLTINSLQPEDFATYYCQHSRELPLTFGGG |
| VH3F | TKLEIKRGGGGSGGGGSGGGGSGGGGSQVQLVQSGAEVKKPGASVKVSCKAS |
| (SEQ ID NO: 96) | GYTFTDYNMDWVRQAPGQGLEWIGDINPNYDSTRYNQKFKGWVTVTRDTSIS |
| | TAYMELSRLTSDDTAVYYCARGDYYVSSYGHDYAMDYWGQGTMVTVSSD |
| VL3E- | DIVLTQSPSSLSASVGDRVTITCRASKSVSTGYSYLHWYQQKPGKAPKLLIY |
| linker- | LASNLESGVPSRFSGSGSGTDFTLTISSLQPEDFATYYCQHSRELPLTFGQG |
| VH3F | TKLEIKRGGGGSGGGGSGGGGSGGGGSQVQLVQSGAEVKKPGASVKVSCKAS |
| (SEQ ID NO: 97) | GYTFTDYNMDWVRQAPGQGLEWIGDINPNYDSTRYNQKFKGWVTVTRDTSIS |
| | TAYMELSRLTSDDTAVYYCARGDYYVSSYGHDYAMDYWGQGTMVTVSSD |
| VL3F- | EIVLTQSPATLSLSPGERATLSCRASKSVSTGYSYLHWYQQKPGQAPRLLIY |
| linker- | LASNLESGVPARFSGSGSGTDFTLTISSLEPEDFAVYYCQHSRELPLTFGGG |
| VH3F | TKVEIKRGGGGSGGGGSGGGGSGGGGSQVQLVQSGAEVKKPGASVKVSCKAS |
| (SEQ ID NO: 98) | GYTFTDYNMDWVRQAPGQGLEWIGDINPNYDSTRYNQKFKGWVTVTRDTSIS |
| | TAYMELSRLTSDDTAVYYCARGDYYVSSYGHDYAMDYWGQGTMVTVSSD |
| VL3G- | DIQMTQSPSSLSASVGDRVTISCRASKSVSTGYSYLHWYQQKPGQAPKLLIY |
| linker- | LASNLESGVPSRFSGSGSGTDFTLTISSLQPEDVATYYCQHSRELPLTFGQG |
| VH3F | TKVEIKRGGGGSGGGGSGGGGSGGGGSQVQLVQSGAEVKKPGASVKVSCKAS |
| (SEQ ID NO: 99) | GYTFTDYNMDWVRQAPGQGLEWIGDINPNYDSTRYNQKFKGWVTVTRDTSIS |
| | TAYMELSRLTSDDTAVYYCARGDYYVSSYGHDYAMDYWGQGTMVTVSSD |
| VL3H- | EIVLTQSPATLSLSIGQRATLSCRASKSVSTGYSYLHWYQQKPGQPPRLLIY |
| linker- | LASNLESGIPARFSGSGPGTDFTLTIRPLEDEDFAVYYCQHSRELPLTFGAG |
| VH3F | TKLELKRGGGGSGGGGSGGGGSGGGGSQVQLVQSGAEVKKPGASVKVSCKAS |
| (SEQ ID NO: 100) | GYTFTDYNMDWVRQAPGOGLEWIGDINPNYDSTRYNQKFKGWVTVTRDTSIS |
| | TAYMELSRLTSDDTAVYYCARGDYYVSSYGHDYAMDYWGQGTMVTVSSD |
| VL3D- | EIVLTQSPSSLSAYVGDRVTITCRASKSVSTGYSYLHWYQQKPGKAPKLLIY |
| linker- | LASNLESGVPSRFSGSGSGTDFTLTINSLQPEDFATYYCQHSRELPLTFGGG |
| VH3G | TKLEIKRGGGGSGGGGSGGGGSGGGGSQVQLVQSGAEVKKPGASVKVSCKAS |
| (SEQ ID NO: 101) | GYTFTDYNMDWVRQAPGQGLEWMGDINPNYDSTRYNQKFKGRVTSTRDTSIS |
| | TAYMELSRLRSDDTVVYYCARGDYYVSSYGHDYAMDYWGQGTSVTVSSD |
| VL3E- | DIVLTQSPSSLSASVGDRVTITCRASKSVSTGYSYLHWYQQKPGKAPKLLIY |
| linker- | LASNLESGVPSRFSGSGSGTDFTLTISSLQPEDFATYYCQHSRELPLTFGQG |
| VH3G | TKLEIKRGGGGSGGGGSGGGGSGGGGSQVQLVQSGAEVKKPGASVKVSCKAS |
| (SEQ ID NO: 102) | GYTFTDYNMDWVRQAPGQGLEWMGDINPNYDSTRYNQKFKGRVTSTRDTSIS |
| | TAYMELSRLRSDDTVVYYCARGDYYVSSYGHDYAMDYWGQGTSVTVSSD |
| VL3F- | EIVLTQSPATLSLSPGERATLSCRASKSVSTGYSYLHWYQQKPGQAPRLLIY |
| linker- | LASNLESGVPARFSGSGSGTDFTLTISSLEPEDFAVYYCQHSRELPLTFGGG |
| VH3G | TKVEIKRGGGGSGGGGSGGGGSGGGGSQVQLVQSGAEVKKPGASVKVSCKAS |
| (SEQ ID NO: 103) | GYTFTDYNMDWVRQAPGQGLEWMGDINPNYDSTRYNQKFKGRVTSTRDTSIS |
| | TAYMELSRLRSDDTVVYYCARGDYYVSSYGHDYAMDYWGQGTSVTVSSD |
| VL3G- | DIQMTQSPSSLSASVGDRVTISCRASKSVSTGYSYLHWYQQKPGQAPKLLIY |
| linker- | LASNLESGVPSRFSGSGSGTDFTLTISSLQPEDVATYYCQHSRELPLTFGQG |
| VH3G | TKVEIKRGGGGSGGGGSGGGGSGGGGSQVQLVQSGAEVKKPGASVKVSCKAS |
| (SEQ ID NO: 104) | GYTFTDYNMDWVRQAPGQGLEWMGDINPNYDSTRYNQKFKGRVTSTRDTSIS |
| | TAYMELSRLRSDDTVVYYCARGDYYVSSYGHDYAMDYWGQGTSVTVSSD |
| VL3H- | EIVLTQSPATLSLSIGQRATLSCRASKSVSTGYSYLHWYQQKPGQPPRLLIY |
| linker- | LASNLESGIPARFSGSGPGTDFTLTIRPLEDEDFAVYYCQHSRELPLTFGAG |
| VH3G | TKLELKRGGGGSGGGGSGGGGSGGGGSQVQLVQSGAEVKKPGASVKVSCKAS |
| (SEQ ID NO: 105) | GYTFTDYNMDWVRQAPGQGLEWMGDINPNYDSTRYNQKFKGRVTSTRDTSIS |
| | TAYMELSRLRSDDTVVYYCARGDYYVSSYGHDYAMDYWGQGTSVTVSSD |
| VL3D- | EIVLTQSPSSLSAYVGDRVTITCRASKSVSTGYSYLHWYQQKPGKAPKLLIY |
| linker- | LASNLESGVPSRFSGSGSGTDFTLTINSLQPEDFATYYCQHSRELPLTFGGG |
| VH3H | TKLEIKRGGGGSGGGGSGGGGSGGGGSEVQLVQSGAELVKPGASVKVSCKAS |
| (SEQ ID NO: 106) | GYTFTDYNMDWVRQSHGRGLEWMGDINPNYDSTRYNQKFKGRVTSTVDKSIS |
| | TAYMELSRLTSDDTVVYYCARGDYYVSSYGHDYAMDYWGQGTSVTVSSD |
| VL3E- | DIVLTQSPSSLSASVGDRVTITCRASKSVSTGYSYLHWYQQKPGKAPKLLIY |
| linker- | LASNLESGVPSRFSGSGSGTDFTLTISSLQPEDFATYYCQHSRELPLTFGQG |
| VH3H | TKLEIKRGGGGSGGGGSGGGGSGGGGSEVQLVQSGAELVKPGASVKVSCKAS |
| (SEQ ID NO: 107) | GYTFTDYNMDWVRQSHGRGLEWMGDINPNYDSTRYNQKFKGRVTSTVDKSIS |
| | TAYMELSRLTSDDTVVYYCARGDYYVSSYGHDYAMDYWGQGTSVTVSSD |
| VL3F- | EIVLTQSPATLSLSPGERATLSCRASKSVSTGYSYLHWYQQKPGQAPRLLIY |
| linker- | LASNLESGVPARFSGSGSGTDFTLTISSLEPEDFAVYYCQHSRELPLTFGGG |
| VH3H | TKVEIKRGGGGSGGGGSGGGGSGGGGSEVQLVQSGAELVKPGASVKVSCKAS |
| (SEQ ID NO: 108) | GYTFTDYNMDWVRQSHGRGLEWMGDINPNYDSTRYNQKFKGRVTSTVDKSIS |
| | TAYMELSRLTSDDTVVYYCARGDYYVSSYGHDYAMDYWGQGTSVTVSSD |
| VL3G- | DIQMTQSPSSLSASVGDRVTISCRASKSVSTGYSYLHWYQQKPGQAPKLLIY |
| linker- | LASNLESGVPSRFSGSGSGTDFTLTISSLQPEDVATYYCQHSRELPLTFGQG |
| VH3H | TKVEIKRGGGGSGGGGSGGGGSGGGGSEVQLVQSGAELVKPGASVKVSCKAS |
| (SEQ ID NO: 109) | GYTFTDYNMDWVRQSHGRGLEWMGDINPNYDSTRYNQKFKGRVTSTVDKSIS |
| | TAYMELSRLTSDDTVVYYCARGDYYVSSYGHDYAMDYWGQGTSVTVSSD |
| VL3H- | EIVLTQSPATLSLSIGQRATLSCRASKSVSTGYSYLHWYQQKPGQPPRLLIY |
| linker- | LASNLESGIPARFSGSGPGTDFTLTIRPLEDEDFAVYYCQHSRELPLTFGAG |
| VH3H | TKLELKRGGGGSGGGGSGGGGSGGGGSEVQLVQSGAELVKPGASVKVSCKAS |
| (SEQ ID NO: 110) | GYTFTDYNMDWVRQSHGRGLEWMGDINPNYDSTRYNQKFKGRVTSTVDKSIS |
| | TAYMELSRLTSDDTVVYYCARGDYYVSSYGHDYAMDYWGQGTSVTVSSD |

In one particular embodiment, the scFv may be VL3H-linker-VH3D, VH3D-linker-VL3H, VL3H-linker-VH3F, VH3F-linker-VL3H, VL3H-linker-VH3H or VH3H-linker-VL3H.

Novel chimeric antigen receptors (CARs) are provided. When the CARs herein are expressed on the surface of immune cells, such immune cells may be used in medicine. In particular, said immune cells may be used in treatment of osteosarcoma. In one embodiment, said immune cells be used in treatment of metastatic osteosarcoma.

As used herein, Chimeric Antigen Receptors (CARs) are artificial receptors comprising an extracellular antigen binding domain, a transmembrane domain and an intracellular signaling domain. The antigen binding domain in CARs is usually a scFv.

CARs in the present disclosure may comprise any of the antigen binding domains as mentioned above. For example, CARs in the present disclosure may comprise any of the scFv's represented by SEQ ID NO: 43 to SEQ ID NO: 76. In particular, the CARs in the present disclosure may comprise any of the antigen binding domains as mentioned above in the form of a scFv which may be connected to a CD8α hinge or a CD28 hinge. In particular, the CARs in the present disclosure may comprise any of the scFv's represented by SEQ ID NO: 43 to SEQ ID NO: 76 connected to a CD8α hinge. In particular, the CARs in the present disclosure may comprise any of the scFv's represented by SEQ ID NO: 43 to SEQ ID NO: 76 connected to a CD8α hinge, wherein the CAR further comprises a transmembrane domain and wherein the intracellular signaling domain comprises a costimulatory domain and a CD3ζ signaling domain.

Alternatively, CARs in the present disclosure may comprise any of the antigen binding domains as mentioned above. For example, CARs in the present disclosure may comprise any of the scFv's represented by SEQ ID NO: 86 to SEQ ID NO: 110. In particular, the CARs in the present disclosure may comprise any of the antigen binding domains as mentioned above in the form of a scFv which may be connected to a CD8α hinge. In particular, the CARs in the present disclosure may comprise any of the scFv's represented by SEQ ID NO: 86 to SEQ ID NO: 110 connected to a CD8α hinge. In particular, the CARs in the present disclosure may comprise any of the scFv's represented by SEQ ID NO: 86 to SEQ ID NO: 110 connected to a CD8α hinge, wherein the CAR further comprises a transmembrane domain and wherein the intracellular signaling domain comprises a costimulatory domain and a CD3ζ signaling domain.

Several hinges derived from human CD8α are known and they may be used in CARs according to the present invention. In general, such hinges contain 10 to 60 amino acid residues from the human CD8α-sequence which in full-length CD8α encodes an extracellular domain connected to the transmembrane domain. In general, such hinges can contain 35 to 55 amino acid residues from the human CD8α-sequence. In general, such hinges can contain 45 to 55 amino acid residues from the human CD8α-sequence. Suitable examples include:

(SEQ ID NO: 116)
FVPVFLPAKPTTTPAPRPPTPAPTIASQPLSLRPEACRPAAGGAVHTRG
LDFACD
and
(SEQ ID NO: 117)
TTTPAPRPPTPAPTIASQPLSLRPEACRPAAGGAVHTRGLDFACDISS

However, the CD8α hinges may comprise 0 to 5 conservative substitutions relative to the human sequence.

The antigen binding domain may be directly attached to a transmembrane domain. However, the CARs may comprise a hinge domain connecting the antigen binding domain to the transmembrane domain. The hinge domain may thus affect the sterical conformation of the antigen binding domain. This may in turn affect the ability of the CAR to bind the target epitope and subsequently trigger signaling into an immune cell. If the target epitope is located too far from the cell membrane of the target cell or if the target epitope is otherwise hidden, the immune cell expressing the CAR may not be efficient. Accordingly, it is preferred that the target epitope is sufficiently accessible for immune cells expressing the CARs.

Four bone-specific isoforms of human tissue-nonspecific alkaline phosphatase (B/I, B1x, B1, and B2) exist according to Haarhaus et al, NATURE REVIEWS—NEPHROLOGY VOLUME 13—JULY 2017. Without being bound by theory, the target antigen of the novel binding units herein may be a bone-specific isoform of human tissue-nonspecific alkaline phosphatase (ALPL), in particular an accessible extracellular epitope thereof. Accordingly, the present disclosure provides CARs comprising a human or humanized scFv targeting a bone-specific isoform of human tissue-nonspecific alkaline phosphatase (e.g. the 524 amino acid residue isoform NM_000478). Cytotoxic immune cells expressing said CARs may be used for treatment of OS with lower risk for triggering unwanted immunogenic responses against the therapeutic cells. In effect, there is also provided a method for treatment of OS comprising a step of administering a pharmaceutical composition intravenously or intra-tumorally to a patient diagnosed with OS, wherein the pharmaceutical composition contains cytotoxic cells expressing a CAR with a human or humanized scFv able to specifically bind to a bone-specific isoform of human tissue-nonspecific alkaline phosphatase. There is also provided CARs comprising a human or humanized scFv able to specifically bind to a bone-specific isoform of human tissue-nonspecific alkaline phosphatase. CARs comprising a human or humanized scFv able to specifically bind to a bone-specific isoform B1 of human tissue-nonspecific alkaline phosphatase may be used for treatment of OS. CARs comprising a human or humanized scFv able to specifically bind to a bone-specific isoform B2 of human tissue-nonspecific alkaline phosphatase may be used for treatment of OS.

In one embodiment, there is provided a method of treatment of OS in a human patient comprising the steps:
a) transducing T cells or NK cells with mRNA encoding any of the CARs herein
b) repeatedly administering an effective dose of a pharmaceutical composition comprising said cells to an OS patient.

The transmembrane domain connects the extracellular domains to an intracellular signaling domain. Both the antigen binding domain and hinge domain are extracellular domains, i.e., that they generally face the extracellular environment when expressed in the cell membrane of an immune cell. As used herein, “transmembrane domain”, means the part of the CAR which tend to be embedded in the cell membrane when expressed by an immune effector cell. Suitable transmembrane domains are well known for skilled persons. In particular, transmembrane domains from the human proteins CD8α, CD28 or ICOS may be used. The transmembrane domain is believed to convey a signal into immune cells upon binding of a target by the antigen binding domain.

The “intracellular signaling domain” refers to a part of the CAR located inside the immune cell when the CAR is expressed in the cell membrane. These domains participate in conveying the signal upon binding of the target. A variety of signaling domains are known, and they can be combined and tailored to fit the endogenous signaling machinery in the immune cells. In one embodiment the intracellular signaling domain comprises a “signal 1” domain like the signaling domains obtainable from the human proteins CD3ζ, FcR-γ, CD3ε etc. In general, it is believed that “signal 1” domains (e.g. CD3ζ signaling domain) convey a signal upon antigen binding.

In another embodiment, the intracellular signaling domain comprises a costimulatory domain. Such domains are well known and often referred to as “signal 2” domains, and they are believed to, subsequently of “signal 1” domains, convey a signal via costimulatory molecules. The “signal 2” is important for the maintenance of the signal and the survival of the cells. If absent, like in first-generation CARs, the CAR T cell may be efficient in killing and in early cytokines release, but it will often become exhausted over time. Examples of such commonly used human “signal 2” domains include 4-1BB signaling domain, CD28 signaling domain, 4-1BB signaling domain and ICOS signaling domain.

The immune cells expressing the CARs herein may be isolated from a patient or a compatible donor by leukapheresis or other suitable methods. Such primary cells may for example be T cells or NK cells. In particular, autologous T cells (both cytotoxic T cells, T helper cells or mixtures of these) may be transduced with nucleic acids encoding the CARs before a pharmaceutical composition comprising the cells is administered back to the patient. The immune cells expressing the CARs may also be cell lines suitable for clinical use like NK-92 cells. Of course, the preferred cells are human when the intended patient is human. However, as OS also affects dogs, canine cells expressing the CARs herein may be used in treatment of OS in dogs.

The pharmaceutical compositions herein can be a composition suitable for administration of therapeutic cells to a patient. The most common administration route for CAR T cells is intravenous administration. Accordingly, said pharmaceutical compositions may for example be sterile aqueous solutions with a neutral pH. Accordingly, said pharmaceutical compositions may for example be sterile aqueous solutions with a physiological pH. For example, a patient's peripheral blood mononuclear cells may be obtained via a standard leukapheresis procedure. The mononuclear cells may be enriched for T cells, before transducing them with a lentiviral vector or mRNA encoding the CARs. Said cells may then be activated with anti-CD3/CD28 antibody coated beads. The transduced T cells may be expanded in cell culture, washed, and formulated into a sterile suspension, which can be cryopreserved. If so, the product is thawed prior to administration.

One issue related to efficient access of CAR T cells at the target site of osteosarcoma, is to circumvent defenses from solid tumors. In contrast to hematological malignancies, recognition of solid tumors requires egress from the blood into the tumor site, and many malignancies evolve such that T cell infiltration is actively impeded. In situations where the tumor is localized, different administration modes may be used to improve efficacy. For example, regional rather than systemic administration of CAR T cells might enhance efficacy.

The pharmaceutical compositions may comprise a pharmaceutically effective dose of the immune cells herein. A pharmaceutically effective dose may for example be in the range of 1×10⁶ to 1×10¹⁰ immune cells expressing the CARs. A pharmaceutically effective dose may for example be in the range of 1×10⁷ to 1×10⁹ T cells expressing the CARs.

For efficient expression of the claimed CARs in immune cells, a conventional leader peptide may be introduced N-terminally for facilitating location in the cell membrane. One example of a leader peptide is METDTLLLWVLLLWVPGSTG (SEQ ID NO: 115). The leader peptide is believed to be trimmed off and will likely not be present in the functional CAR in the cell membrane.

Accordingly, for expression of a second-generation CAR, nucleic acids encoding the following may be used:

N-LEADER PEPTIDE-VH-LINKER-VL-HINGE-TRANSMEMBRANE DOMAIN-COSTIMULATORY DOMAIN-SIGNALING DOMAIN.

Accordingly, for expression of a second-generation CAR, nucleic acids encoding the following may also be used:

N-LEADER PEPTIDE-VL-LINKER-VH-HINGE-TRANSMEMBRANE DOMAIN-COSTIMULATORY DOMAIN-SIGNALING DOMAIN

The nucleic acids encoding the claimed CARs can be in the form of well-known RNA e.g. mRNA, or DNA expression vectors.

The murine parent VL TP1 (SEQ ID NO: 111) with CDRs boxed:

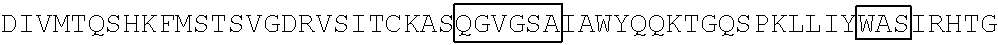

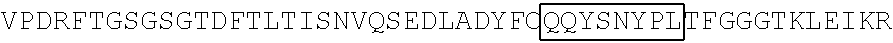

The murine parent VH TP1 (SEQ ID NO: 112) with CDRs boxed:

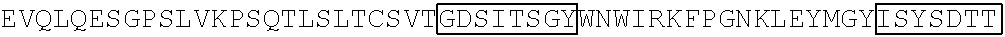

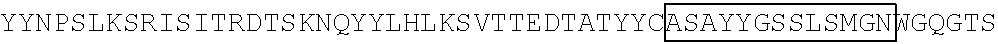

ATVSSD
The murine parent VL TP3 (SEQ ID NO: 113) with CDRs boxed:

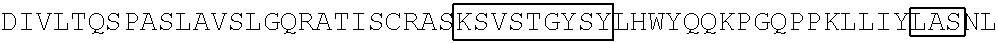

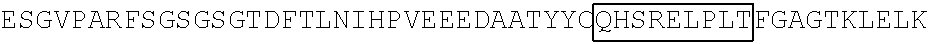

The murine parent VH TP3 (SEQ ID NO: 114) with CDRs boxed:

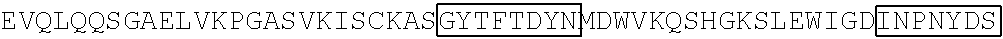

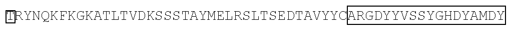

WGQGTSVTVSS

It has previously been shown in PCT/EP2019/086309 that CARs comprising murine VL TP1 (SEQ ID NO: 111) and murine VH TP1 (SEQ ID NO: 112) provides therapeutic effect and prolonged survival in mice with very aggressive OSA lung tumors. It was further shown in PCT/EP2019/086309 that the CARs have cytotoxic effect on tumor cells (OHS cell line).

The murine VL TP1 (SEQ ID NO: 111) and murine VH TP1 (SEQ ID NO: 112) comprises the same 6 CDR's as provided by the present invention and as represented by SEQ ID NO: 1 to 5 as shown below.

In certain embodiments it is thus expected that a protein for targeting of osteosarcoma cells, comprising a VL and a VH which together form an antigen binding unit according to the present invention, wherein the VL comprises three complementarity determining regions (CDRs); CDR1, CDR2 and CDR3 which respectively are represented by the amino acid sequences SEQ ID NO: 1, WAS and SEQ ID NO: 2 and wherein the VH comprises three CDRs; CDR1, CDR2 and CDR3 which respectively are represented by the amino acid sequences SEQ ID NOs: 3, 4 and 5, and wherein all the CDRs are flanked by human framework sequences, and optionally wherein each of the human framework sequences, independently, comprises 0 to 5 amino acid substitutions have a similar effect as described in PCT/EP2019/086309 such as cytotoxic effect on osteosarcoma cells.

In certain embodiments it is further expected that a CAR construct comprising the above antigen binding unit and according to the present invention have a similar effect as described in PCT/EP2019/086309 such as cytotoxic effect on osteosarcoma cells.

It has also previously been shown in PCT/EP2019/086309 that CARs comprising murine VL TP3 (SEQ ID NO: 113) and murine VH TP3 (SEQ ID NO: 114) provides therapeutic effect and prolonged survival in mice with very aggressive OSA lung tumors. It was further shown in PCT/EP2019/086309 that the CARs have cytotoxic effect on tumor cells (OHS cell line).

The murine VL TP3 (SEQ ID NO: 113) and murine VH TP3 (SEQ ID NO: 114) comprises the same 6 CDR's as provided by the present invention and as represented by SEQ ID NO: 5 to 10 as shown below.

In certain embodiments it is thus expected that a protein for targeting of osteosarcoma cells, comprising a VL and a VH which together form an antigen binding unit according to the present invention, wherein the VL comprises three complementarity determining regions (CDRs); CDR1, CDR2 and CDR3 which respectively are represented by the amino acid sequences SEQ ID NO: 6, LAS and SEQ ID NO: 7 and wherein the VH comprises three CDRs; CDR1, CDR2 and CDR3 which respectively are represented by the amino acid sequences SEQ ID NOs: 8, 9 and 10, and wherein all the CDRs are flanked by human framework sequences, and optionally wherein each of the human framework sequences, independently, comprises 0 to 5 amino acid substitutions have a similar effect as described in PCT/EP2019/086309 such as cytotoxic effect on osteosarcoma cells.

In certain embodiments it is further expected that a CAR construct comprising the above antigen binding unit and according to the present invention have a similar effect as described in PCT/EP2019/086309 such as cytotoxic effect on osteosarcoma cells.

SEQUENCES

TP1 VL CDRs
SEQ ID NO: 1 QGVGSA
SEQ ID NO: 2 QQYSNYPLT
TP1 VH CDRs
SEQ ID NO: 3 GDSITSGY
SEQ ID NO: 4 ISYSDTT
SEQ ID NO: 5 ASAYYGSSLSMGN
TP3 VL CDRs
SEQ ID NO: 6 SKSVSTGYSY
SEQ ID NO: 7 QHSRELPLT
TP3 VH CDRs
SEQ ID NO: 8 GYTFTDYN
SEQ ID NO: 9 INPNYDST
SEQ ID NO: 10 ARGDYYVSSYGHDYAMDY
VL's TP1
SEQ ID NO: 11 (VL1A)
SEQ ID NO: 12 (VL1B)
SEQ ID NO: 13 (VL1C)
SEQ ID NO: 14 (VL1D)
SEQ ID NO: 15 (VL1E)
SEQ ID NO: 16 (VL1F)
SEQ ID NO: 17 (VL1G)
SEQ ID NO: 18 (VL1H)
VH's TP1
SEQ ID NO: 19 (VH1A)
SEQ ID NO: 20 (VH1B)
SEQ ID NO: 21 (VH1C)
SEQ ID NO: 22 (VH1D)
SEQ ID NO: 23 (VH1E)
SEQ ID NO: 24 (VH1F)
SEQ ID NO: 25 (VH1G)
SEQ ID NO: 26 (VH1H)
VL's TP3
SEQ ID NO: 27 (VL3A)
SEQ ID NO: 28 (VL3B)
SEQ ID NO: 29 (VL3C)
SEQ ID NO: 30 (VL3D)
SEQ ID NO: 31 (VL3E)
SEQ ID NO: 32 (VL3F)
SEQ ID NO: 33 (VL3G)
SEQ ID NO: 34 (VL3H)
VH's TP3
SEQ ID NO: 35 (VH3A)
SEQ ID NO: 36 (VH3B)
SEQ ID NO: 37 (VH3C)
SEQ ID NO: 38 (VH3D)
SEQ ID NO: 39 (VH3E)
SEQ ID NO: 40 (VH3F)
SEQ ID NO: 41 (VH3G)
SEQ ID NO: 42 (VH3H)
scFv's TP1:
SEQ ID NO: 43 VL1A-linker-VH1A
SEQ ID NO: 44 VL1A-linker-VH1B
SEQ ID NO: 45 VL1A-linker-VH1C
SEQ ID NO: 46 VL1B-linker-VH1A
SEQ ID NO: 47 VL1B-linker-VH1B
SEQ ID NO: 48 VL1B-linker-VH1C
SEQ ID NO: 49 VL1C-linker-VH1A
SEQ ID NO: 50 VL1C-linker-VH1B
SEQ ID NO: 51 VL1C-linker-VH1C
SEQ ID NO: 52 VL1D-linker-VH1D
SEQ ID NO: 53 VL1E-linker-VH1D
SEQ ID NO: 54 VL1F-linker-VH1D
SEQ ID NO: 55 VL1G-linker-VH1D
SEQ ID NO: 56 VL1H-linker-VH1D
SEQ ID NO: 57 VL1D-linker-VH1E
SEQ ID NO: 58 VL1E-linker-VH1E
SEQ ID NO: 59 VL1F-linker-VH1E
SEQ ID NO: 60 VL1G-linker-VH1E
SEQ ID NO: 61 VL1H-linker-VH1E
SEQ ID NO: 62 VL1D-linker-VH1F
SEQ ID NO: 63 VL1E-linker-VH1F
SEQ ID NO: 64 VL1F-linker-VH1F
SEQ ID NO: 65 VL1G-linker-VH1F
SEQ ID NO: 66 VL1H-linker-VH1F
SEQ ID NO: 67 VL1D-linker-VH1G
SEQ ID NO: 68 VL1E-linker-VH1G
SEQ ID NO: 69 VL1F-linker-VH1G
SEQ ID NO: 70 VL1G-linker-VH1G
SEQ ID NO: 71 VL1H-linker-VH1G
SEQ ID NO: 72 VL1D-linker-VH1H
SEQ ID NO: 73 VL1E-linker-VH1H
SEQ ID NO: 74 VL1F-linker-VH1H
SEQ ID NO: 75 VL1G-linker-VH1H
SEQ ID NO: 76 VL1H-linker-VH1H
scFv's TP3:
SEQ ID NO: 77 VL3A-linker-VH3A
SEQ ID NO: 78 VL3A-linker-VH3B
SEQ ID NO: 79 VL3A-linker-VH3C
SEQ ID NO: 80 VL3B-linker-VH3A
SEQ ID NO: 81 VL3B-linker-VH3B
SEQ ID NO: 82 VL3B-linker-VH3C
SEQ ID NO: 83 VL3C-linker-VH3A
SEQ ID NO: 84 VL3C-linker-VH3B
SEQ ID NO: 85 VL3C-linker-VH3C
scFv's:
SEQ ID NO: 86 VL3D-linker-VH3D
SEQ ID NO: 87 VL3E-linker-VH3D
SEQ ID NO: 88 VL3F-linker-VH3D
SEQ ID NO: 89 VL3G-linker-VH3D
SEQ ID NO: 90 VL3H-linker-VH3D
SEQ ID NO: 91 VL3D-linker-VH3E
SEQ ID NO: 92 VL3E-linker-VH3E
SEQ ID NO: 93 VL3F-linker-VH3E
SEQ ID NO: 94 VL3G-linker-VH3E
SEQ ID NO: 95 VL3H-linker-VH3E
SEQ ID NO: 96 VL3D-linker-VH3F
SEQ ID NO: 97 VL3E-linker-VH3F
SEQ ID NO: 98 VL3F-linker-VH3F
SEQ ID NO: 99 VL3G-linker-VH3F
SEQ ID NO: 100 VL3H-linker-VH3F
SEQ ID NO: 101 VL3D-linker-VH3G
SEQ ID NO: 102 VL3E-linker-VH3G
SEQ ID NO: 103 VL3F-linker-VH3G
SEQ ID NO: 104 VL3G-linker-VH3G
SEQ ID NO: 105 VL3H-linker-VH3G
SEQ ID NO: 106 VL3D-linker-VH3H
SEQ ID NO: 107 VL3E-linker-VH3H
SEQ ID NO: 108 VL3F-linker-VH3H
SEQ ID NO: 109 VL3G-linker-VH3H
SEQ ID NO: 110 VL3H-linker-VH3H
Parent:
SEQ ID NO: 111 murine parent VL TP1
SEQ ID NO: 112 murine parent VH TP1
SEQ ID NO: 113 murine parent VL TP3
SEQ ID NO: 114 murine parent VH TP3
Leader peptide
SEQ ID NO: 115 leader peptide

### EXAMPLES

### Example 1

Novel second-generation murine CARs and humanized CARs can be cloned into pENTR™ vector (Invitrogen) and further subcloned by recombination into Gateway system compatible expression vectors, the retroviral construct pMP71 and the mRNA synthesis construct pCIpA102 following our previous publication (Wälchli et al, PLoS One. 2011; 6(11):e27930).

The T cell line Jurkat-76 constitutively expressing NFAT-GFP reporter gene (Jutz S, Leitner J, Schmetterer K, Doel-Perez I, Majdic O, Grabmeier-Pfistershammer K, Paster W, Huppa J B, Steinberger P. J Immunol Methods. 2016 Jan. 15. pii: S0022-1759(16)30007-2. doi: 10.1016/j.jim.2016.01.007. 10.1016/j.jim.2016.01.007 PubMed 26780292) were transduced by retroviral particles encoding the humanized CARs via well-known methods (Wälchli et al, PLoS One. 2011; 6(11):e27930). After 48 hours, the presence of the CAR can be analyzed by flow cytometry.

In vitro recognition of target the OSA cell line can be demonstrated by incubating J76-NFAT-GFP-CARs with OSA for 20 hours. If the TCR signaling pathway is stimulated, the NFAT promoter becomes activated and GFP is produced into the cytosol of J76-NFAT-GFP cells. These cells can be analyzed by flow cytometry and the level of GFP can be quantified, it will be proportional to the level of stimulation. This assay can be run with different cell lines, OS cell lines or healthy cells and is sensitive enough to detect if the CAR's recognition is restricted to OS. It also informs us about the tonic signal of certain constructs.

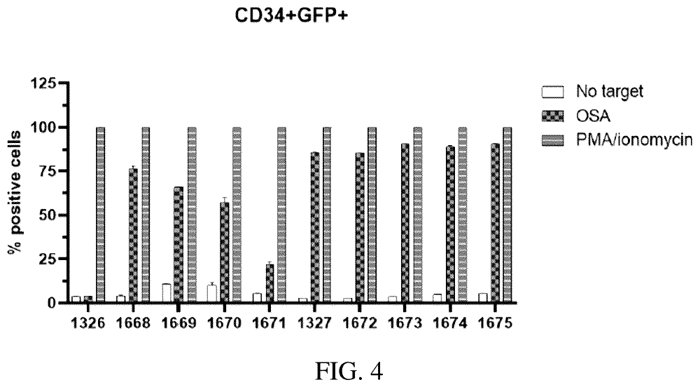

In vitro activation by target cell lines was demonstrated by incubating J76-NFAT expressing the humanized CARs with osteosarcoma cell line (OSA) for 24 hours as described in example 1, above. The results are depicted in FIG. 4. Clone 1326 (OSCAR-1) and clone 1327 (OSCAR-3) are second generation CARs based on murine sequences. Clone 1668, 1669, 1670 and 1671 are humanized variants of TP1/OSCAR 1, while clone 1672, 1673, 1674 and 1675 are humanized variants of TP3/OSCAR 3. PMA/ionomycin is a positive control for normalization of expression data between samples. The results demonstrates that humanization of TP3 scFv does not affect the activity/binding activity of the CAR constructs.

### Example 2

In vitro killing of target cells is performed using primary human T cells isolated from healthy donor. The T cells are genetically modified using retrovirus as mentioned above and further expanded. Target cells are also genetically modified with a transgene encoding for a luciferase-GFP fusion protein (Loew, R., Heinz, N., Hampf, M. et al. Improved Tet-responsive promoters with minimized background expression. BMC Biotechnol 10, 81 (2010).). Here, the luciferase will be constitutively produced and its activity detectable upon addition of its substrate, luciferin. As long as the cells are alive they will produce luciferase that will emit detectable light signals, upon cell death, the luciferin will not be metabolized due to the lack of ATP and the signal will be lost. The quantification of the signal will be used to quantify cell death. CAR T cells are co-cultured with target cells positive or negative for the antigen of interest at different Effector-to-Target (E:T) ratio and the luciferase signal will be monitored at different time points and plotted.

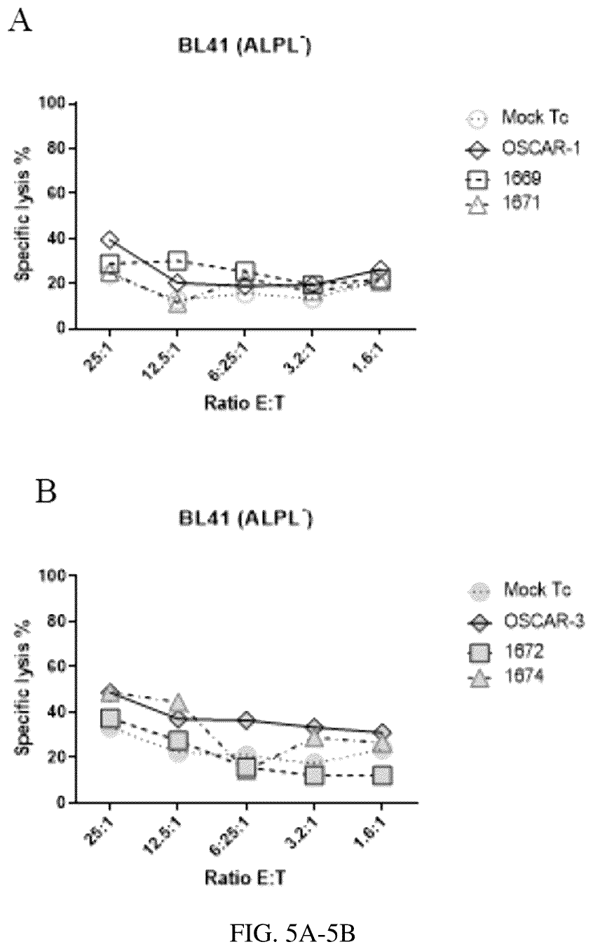

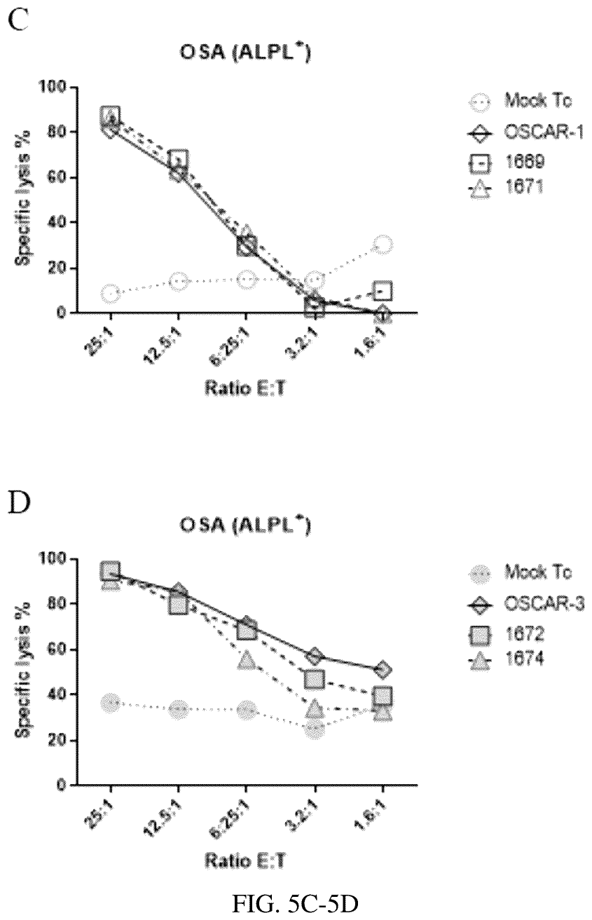

As shown in FIG. 5, cells expressing OSCAR-1 and OSCAR-3 strictly kill OSA target cells, suggesting that they are specific. In addition, cells expressing the humanized constructs (1669/1671 and 1672/1674) specifically kill OSA target cells with the similar efficiency, suggesting that the functional avidity of the humanized constructs is conserved.

The different clones as described above refer to second generation CARs varying with respect to the scFv only:

| Clone | scFv | SEQ ID NO |
|---|---|---|
| 1668 | VL1A-linker-VH1A | 43 |
| 1669 | VL1A-linker-VH1B | 44 |
| 1670 | VL1B-linker-VH1A | 45 |
| 1671 | VL1B-linker-VH1B | 47 |
| 1672 | VL3A-linker-VH3A | 77 |
| 1673 | VL3A-linker-VH3B | 78 |
| 1674 | VL3A-linker-VH3C | 79 |
| 1675 | VL3B-linker-VH3A | 80 |

The structure of the tested CARs herein can be represented: scFv-CD8α hinge-CD8α transmembrane domain-4-1BB costimulatory domain-CD3ζ signaling domain.

In order to assess CAR efficacy, an animal model can be used in which human osteosarcoma cell lines are engrafted in mice. The engraftment can be performed by intra-peritoneal injection of 10⁶ OHS cells expressing the luciferase gene. When the tumor become palpable, the mice can be randomized and treated or not with T cells redirected or not with the novel CARs. Three injections of 10×10⁶ T cells can be administered and the tumor growth can be monitored by IVIS (luciferase signal detection).

1. A protein for targeting of osteosarcoma cells, comprising a VL and a VH which together form an antigen binding unit,
wherein the VL comprises three complementarity determining regions (CDRs); CDR1, CDR2 and CDR3 which respectively are represented by the amino acid sequences SEQ ID NO: 1, WAS and SEQ ID NO: 2 and
wherein the VH comprises three CDRs; CDR1, CDR2 and CDR3 which respectively are represented by the amino acid sequences SEQ ID NOs: 3, 4 and 5, and
wherein all the CDRs are flanked by human framework sequences, and optionally wherein each of the human framework sequences, independently, comprises 0 to 5 amino acid substitutions.

2. The protein according to claim 1, wherein the VL is selected from the group consisting of VL1 A (SEQ ID NO: 11), VL1B (SEQ ID NO: 12), VL1C (SEQ ID NO: 13), VL1D (SEQ ID NO: 14), VL1E (SEQ ID NO: 15), VL1F (SEQ ID NO: 16), VL1G (SEQ ID NO:17) and VL1H (SEQ ID NO: 18), wherein the VH is selected from the group consisting of VH1A (SEQ ID NO: 19), VH1B (SEQ ID NO: 20), VH1C (SEQ ID NO: 21), VH1D (SEQ ID NO: 22), VH1E (SEQ ID NO: 23), VH1F (SEQ ID NO: 24), VH1G (SEQ ID NO: 25) and VH1H (SEQ ID NO: 26) and wherein the antigen binding unit is selected from the scFv's represented by SEQ ID NO: 43 to SEQ ID NO: 76.

3. (canceled)

4. A Chimeric Antigen Receptor (CAR) comprising the protein according to claim 1.

5. (canceled)

6. (canceled)

7. An immune cell expressing a CAR according to claim 4 in its cell membrane.

8. A pharmaceutical composition comprising the protein according to claim 1.

9. A pharmaceutical composition comprising a cytotoxic immune cell according to claim 7.

10. A method of treatment of osteosarcoma in a human patient comprising the step of administering to the human patient the pharmaceutical composition according to claim 9.

11. An antibody comprising the protein according to claim 1.

12. (canceled)

13. A method for treatment of osteosarcoma in a human patient comprising the step of administering a pharmaceutical composition comprising the antibody of claim 11.

14. An in vitro diagnostic method comprising detecting the antibody according to claim 11.

15. A protein for targeting of osteosarcoma cells, comprising a VL and a VH which together form an antigen binding unit,
wherein the VL comprises three complementarity determining regions (CDRs); CDR1, CDR2 and CDR3 which respectively are represented by the amino acid sequences SEQ ID NO: 6, LAS and SEQ ID NO: 7 and
wherein the VH comprises three CDRs; CDR1, CDR2 and CDR3 which respectively are represented by the amino acid sequences SEQ ID NOs: 8, 9 and 10, and
wherein all the CDRs are flanked by human framework sequences, and optionally wherein each of the human framework sequences, independently, comprises 0 to 5 amino acid substitutions.

16. The protein according to claim 15, wherein the VL is selected from the group consisting of VL3A (SEQ ID NO: 27), VL3B (SEQ ID NO: 28), VL3C (SEQ ID NO: 29), VL3D (SEQ ID NO: 30), VL3E (SEQ ID NO: 31), VL3F (SEQ ID NO: 32), VL3G (SEQ ID NO:33) and VL3H (SEQ ID NO: 34), wherein the VH is selected from the group consisting of VH3A (SEQ ID NO: 35), VH3B (SEQ ID NO: 36), VH3C (SEQ ID NO: 37), VH3D (SEQ ID NO: 38), VH3E (SEQ ID NO: 39), VH3F (SEQ ID NO: 40), VH3G (SEQ ID NO: 41) and VH3H (SEQ ID NO: 42), and wherein the antigen binding unit is selected from the scFv's represented by SEQ ID NO: 77 to SEQ ID NO: 110.

17. (canceled)

18. A Chimeric Antigen Receptor (CAR) comprising the protein according to claim 15.

19. (canceled)

20. (canceled)

21. An immune cell expressing the CAR according to claim 18 in its cell membrane.

22. A pharmaceutical composition comprising the protein according to claim 15.

23. A pharmaceutical composition comprising a cytotoxic immune cell according to claim 21.

24. A method of treatment of osteosarcoma in a human patient comprising the step of administering to the human patient the pharmaceutical composition according to claim 23.

25. An antibody comprising the protein according to claim 15.

26. (canceled)

27. A method for treatment of osteosarcoma in a human patient comprising the step of administering to the human patient a pharmaceutical composition comprising the antibody according to claim 25.

28. An in vitro diagnostic method comprising detecting the antibody according to claim 25.


SEQUENCE LISTING (115 sequences)

SEQ ID NO: 1 - protein, 6 aa, Artificial Sequence
  TP1 VL CDR 1
QGVGSA

SEQ ID NO: 2 - protein, 9 aa, Artificial Sequence
  TP1 VL CDR3
QQYSNYPLT

SEQ ID NO: 3 - protein, 8 aa, Artificial Sequence
  TP1 VH CDR1
GDSITSGY

SEQ ID NO: 4 - protein, 7 aa, Artificial Sequence
  TP1 VH CDR2
ISYSDTT

SEQ ID NO: 5 - protein, 13 aa, Artificial Sequence
  TP1 VH CDR3
ASAYYGSSLSMGN

SEQ ID NO: 6 - protein, 10 aa, Artificial Sequence
  TP3 VL CDR1
SKSVSTGYSY

SEQ ID NO: 7 - protein, 9 aa, Artificial Sequence
  TP3 VL CDR 3
QHSRELPLT

SEQ ID NO: 8 - protein, 8 aa, Artificial Sequence
  TP3 VH CDR1
GYTFTDYN

SEQ ID NO: 9 - protein, 8 aa, Artificial Sequence
  TP3 VH CDR2
INPNYDST

SEQ ID NO: 10 - protein, 18 aa, Artificial Sequence
  TP3 VH CDR 3
ARGDYYVSSYGHDYAMDY

SEQ ID NO: 11 - protein, 108 aa, Artificial Sequence
  TP1 VL1A
DIVMTQSPSFLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKR

SEQ ID NO: 12 - protein, 108 aa, Artificial Sequence
  TP1 VL1B
DIVMTQSPSSLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKR

SEQ ID NO: 13 - protein, 108 aa, Artificial Sequence
  TP1 VL1C
DIVMTQSPSSVSASVGDRVTITCKASQGVGSAIAWYQQKPGQSPKLLIYWASIRHTGVPDRFTGSGSGTDFTLTISSLQPEDFATYFCQQYSNYPLTFGGGTKLTVKR

SEQ ID NO: 14 - protein, 107 aa, Artificial Sequence
  TP1 VL1D
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIK

SEQ ID NO: 15 - protein, 107 aa, Artificial Sequence
  TP1 VL1E
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIK

SEQ ID NO: 16 - protein, 107 aa, Artificial Sequence
  TP1 VL1F
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIK

SEQ ID NO: 17 - protein, 107 aa, Artificial Sequence
  TP1 VL1G
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIK

SEQ ID NO: 18 - protein, 107 aa, Artificial Sequence
  TP1 VL1H
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIK

SEQ ID NO: 19 - protein, 119 aa, Artificial Sequence
  TP1 VH1A
EVQLQESGPGLVKPSETLSLTCSVTGDSITSGYWNWIRQPPGNKLEYMGYISYSDTTYYNPSLKSRITITRDTSKNQYYLKLSSVTAADTATYYCASAYYGSSLSMGNWGQGTTVTVSS

SEQ ID NO: 20 - protein, 119 aa, Artificial Sequence
  TP1 VH1B
EVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTTVTVSS

SEQ ID NO: 21 - protein, 119 aa, Artificial Sequence
  TP1 VH1C
EVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTLVTVSS

SEQ ID NO: 22 - protein, 119 aa, Artificial Sequence
  TP1 VH1D
QLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSS

SEQ ID NO: 23 - protein, 119 aa, Artificial Sequence
  TP1 VH1E
QVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSS

SEQ ID NO: 24 - protein, 119 aa, Artificial Sequence
  TP1 VH1F
EVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSS

SEQ ID NO: 25 - protein, 119 aa, Artificial Sequence
  TP1 VH1G
QVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSS

SEQ ID NO: 26 - protein, 119 aa, Artificial Sequence
  TP1 VH1H
DVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSS

SEQ ID NO: 27 - protein, 111 aa, Artificial Sequence
  TP3 VL3A
DIVLTQSPDSLAVSLGERATISCRASKSVSTGYSYLHWYQQKPGQPPKLLIYLASNLESGVPARFSGSGSGTDFTLTISSLQAEDVAVYYCQHSRELPLTFGAGTKLEIKR

SEQ ID NO: 28 - protein, 111 aa, Artificial Sequence
  TP3 VL3B
DIVLTQSPATLSLSPGERATLSCRASKSVSTGYSYLHWYQQKPGQPPKLLIYLASNLESGVPARFSGSGSGTDFTLTISSLEPEDFAVYYCQHSRELPLTFGAGTKLEIKR

SEQ ID NO: 29 - protein, 111 aa, Artificial Sequence
  TP3 VL3C
DIVLTQSPATLSVSPGERATLSCRASKSVSTGYSYLHWYQQKPGQPPKLLIYLASNLESGVPARFSGSGSGTDFTLTISSLQSEDFAVYYCQHSRELPLTFGAGTKVEIKR

SEQ ID NO: 30 - protein, 110 aa, Artificial Sequence
  TP3 VL3D
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIK

SEQ ID NO: 31 - protein, 110 aa, Artificial Sequence
  TP3 VL3E
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIK

SEQ ID NO: 32 - protein, 110 aa, Artificial Sequence
  TP3 VL3F
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIK

SEQ ID NO: 33 - protein, 110 aa, Artificial Sequence
  TP3 VL3G
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIK

SEQ ID NO: 34 - protein, 110 aa, Artificial Sequence
  TP3 VL3H
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELK

SEQ ID NO: 35 - protein, 125 aa, Artificial Sequence
  TP3 VH3A
EVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 36 - protein, 125 aa, Artificial Sequence
  TP3 VH3B
EVQLQQSGAEVKKPGASVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 37 - protein, 125 aa, Artificial Sequence
  TP3 VH3C
EVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTLVTVSS

SEQ ID NO: 38 - protein, 125 aa, Artificial Sequence
  TP3 VH3D
QVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSS

SEQ ID NO: 39 - protein, 125 aa, Artificial Sequence
  TP3 VH3E
QVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSS

SEQ ID NO: 40 - protein, 125 aa, Artificial Sequence
  TP3 VH3F
QVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSS

SEQ ID NO: 41 - protein, 125 aa, Artificial Sequence
  TP3 VH3G
QVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSS

SEQ ID NO: 42 - protein, 125 aa, Artificial Sequence
  TP3 VH3H
EVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSS

SEQ ID NO: 43 - protein, 247 aa, Artificial Sequence
  VL1A-linker-VH1A
DIVMTQSPSFLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSEVQLQESGPGLVKPSETLSLTCSVTGDSITSGYWNWIRQPPGNKLEYMGYISYSDTTYYNPSLKSRITITRDTSKNQYYLKLSSVTAADTATYYCASAYYGSSLSMGNWGQGTTVTVSS

SEQ ID NO: 44 - protein, 247 aa, Artificial Sequence
  VL1A-linker-VH1B
DIVMTQSPSFLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTTVTVSS

SEQ ID NO: 45 - protein, 247 aa, Artificial Sequence
  VL1A-linker-VH1C
DIVMTQSPSFLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTLVTVSS

SEQ ID NO: 46 - protein, 236 aa, Artificial Sequence
  VL1B-linker-VH1A
DIVMTQSPSSLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSDIVMTQSPSSLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKR

SEQ ID NO: 47 - protein, 247 aa, Artificial Sequence
  VL1B-linker-VH1B
DIVMTQSPSSLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTTVTVSS

SEQ ID NO: 48 - protein, 247 aa, Artificial Sequence
  VL1B-linker-VH1C
DIVMTQSPSSLSASVGDRVTITCKASQGVGSAIAWYQQKPGQSPKLLIYWASIRHTGVPDRFTGSGSGTDFTLTISSLQPEDFATYFCQQYSNYPLTFGGGTKVEIKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTLVTVSS

SEQ ID NO: 49 - protein, 247 aa, Artificial Sequence
  VL1C-linker-VH1A
DIVMTQSPSSVSASVGDRVTITCKASQGVGSAIAWYQQKPGQSPKLLIYWASIRHTGVPDRFTGSGSGTDFTLTISSLQPEDFATYFCQQYSNYPLTFGGGTKLTVKRGGGGSGGGGSGGGGSGGGGSEVQLQESGPGLVKPSETLSLTCSVTGDSITSGYWNWIRQPPGNKLEYMGYISYSDTTYYNPSLKSRITITRDTSKNQYYLKLSSVTAADTATYYCASAYYGSSLSMGNWGQGTTVTVSS

SEQ ID NO: 50 - protein, 247 aa, Artificial Sequence
  VL1C-linker-VH1B
DIVMTQSPSSVSASVGDRVTITCKASQGVGSAIAWYQQKPGQSPKLLIYWASIRHTGVPDRFTGSGSGTDFTLTISSLQPEDFATYFCQQYSNYPLTFGGGTKLTVKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTTVTVSS

SEQ ID NO: 51 - protein, 247 aa, Artificial Sequence
  VL1C-linker-VH1C
DIVMTQSPSSVSASVGDRVTITCKASQGVGSAIAWYQQKPGQSPKLLIYWASIRHTGVPDRFTGSGSGTDFTLTISSLQPEDFATYFCQQYSNYPLTFGGGTKLTVKRGGGGSGGGGSGGGGSGGGGSEVQLQESGPGLVKPSQTLSLTCSVTGDSITSGYWNWIRQHPGNKLEYMGYISYSDTTYYNPSLKSRITITRDTSKNQYSLKLSSVTAADTATYYCASAYYGSSLSMGNWGQGTLVTVSS

SEQ ID NO: 52 - protein, 248 aa, Artificial Sequence
  VL1D-linker-VH1D
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIKRGGGGSGGGGSGGGGSGGGGSQLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSSD

SEQ ID NO: 53 - protein, 248 aa, Artificial Sequence
  VL1E-linker-VH1D
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIKRGGGGSGGGGSGGGGSGGGGSQLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSSD

SEQ ID NO: 54 - protein, 248 aa, Artificial Sequence
  VL1F-linker-VH1D
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIKRGGGGSGGGGSGGGGSGGGGSQLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSSD

SEQ ID NO: 55 - protein, 248 aa, Artificial Sequence
  VL1G-linker-VH1D
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIKRGGGGSGGGGSGGGGSGGGGSQLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSSD

SEQ ID NO: 56 - protein, 248 aa, Artificial Sequence
  VL1H-linker-VH1D
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIKRGGGGSGGGGSGGGGSGGGGSQLQLQESGPGLVKPSQTLSLTCSVSGDSITSGYWNWIRQHPGKGLEWIGYISYSDTTYYNPSLKSRITISLDTSKNQFSLKLTSVTAADTALYYCASAYYGSSLSMGNWGRGTLVTVSSD

SEQ ID NO: 57 - protein, 248 aa, Artificial Sequence
  VL1D-linker-VH1E
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIKRGGGGSGGGGSGGGGSGGGGSQVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSSD

SEQ ID NO: 58 - protein, 248 aa, Artificial Sequence
  VL1E-linker-VH1E
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIKRGGGGSGGGGSGGGGSGGGGSQVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSSD

SEQ ID NO: 59 - protein, 248 aa, Artificial Sequence
  VL1F-linker-VH1E
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIKRGGGGSGGGGSGGGGSGGGGSQVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSSD

SEQ ID NO: 60 - protein, 248 aa, Artificial Sequence
  VL1G-linker-VH1E
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIKRGGGGSGGGGSGGGGSGGGGSQVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSSD

SEQ ID NO: 61 - protein, 248 aa, Artificial Sequence
  VL1H-linker-VH1E
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIKRGGGGSGGGGSGGGGSGGGGSQVQLQESGPGLVKPSQTLSLTCTVSGDSITSGYWNWIRQPPGKGLEWIGYISYSDTTYYNPSLKSRISISLDTSKNQFSLRLSSVTAADTAVYYCASAYYGSSLSMGNWGQGTTVTVSSD

SEQ ID NO: 62 - protein, 248 aa, Artificial Sequence
  VL1D-linker-VH1F
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIKRGGGGSGGGGSGGGGSGGGGSEVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSSD

SEQ ID NO: 63 - protein, 248 aa, Artificial Sequence
  VL1E-linker-VH1F
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIKRGGGGSGGGGSGGGGSGGGGSEVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSSD

SEQ ID NO: 64 - protein, 248 aa, Artificial Sequence
  VL1F-linker-VH1F
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIKRGGGGSGGGGSGGGGSGGGGSEVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSSD

SEQ ID NO: 65 - protein, 248 aa, Artificial Sequence
  VL1G-linker-VH1F
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIKRGGGGSGGGGSGGGGSGGGGSEVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSSD

SEQ ID NO: 66 - protein, 248 aa, Artificial Sequence
  VL1H-linker-VH1F
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIKRGGGGSGGGGSGGGGSGGGGSEVQLVESGPGLVKPSQTLSLTCTVSGDSITSGYWNWIRQHPGKGLEWIGYISYSDTTYYNPSLKSRVTISVDTSKNQFSLKLSSVTAADTAVYYCASAYYGSSLSMGNWGQGTLVTVSSD

SEQ ID NO: 67 - protein, 248 aa, Artificial Sequence
  VL1D-linker-VH1G
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIKRGGGGSGGGGSGGGGSGGGGSQVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSSD

SEQ ID NO: 68 - protein, 248 aa, Artificial Sequence
  VL1E-linker-VH1G
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIKRGGGGSGGGGSGGGGSGGGGSQVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSSD

SEQ ID NO: 69 - protein, 248 aa, Artificial Sequence
  VL1F-linker-VH1G
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIKRGGGGSGGGGSGGGGSGGGGSQVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSSD

SEQ ID NO: 70 - protein, 248 aa, Artificial Sequence
  VL1G-linker-VH1G
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIKRGGGGSGGGGSGGGGSGGGGSQVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSSD

SEQ ID NO: 71 - protein, 248 aa, Artificial Sequence
  VL1H-linker-VH1G
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIKRGGGGSGGGGSGGGGSGGGGSQVQLQESGPGLVKPSQTLSLTCTVSGDSITSGYWNWIRRFPGNKLEYIGYISYSDTTYYNPSLKSRVTISRDTSKNQFSLKLSSVTAEDTASYYCASAYYGSSLSMGNWGQGTLVTVSSD

SEQ ID NO: 72 - protein, 248 aa, Artificial Sequence
  VL1D-linker-VH1H
EIVLTQSPSSVSASVGDRVTITCKASQGVGSAIAWYQQKPGKAPKLLIYWASIRHTGVPSRFSGSGSGTDFTLTISSLQPEDFATYYCQQYSNYPLTFGGGTKVEIKRGGGGSGGGGSGGGGSGGGGSDVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSSD

SEQ ID NO: 73 - protein, 248 aa, Artificial Sequence
  VL1E-linker-VH1H
DIVMTQSPSTLSASVGDRVTITCKASQGVGSAIAWYQQKPGKAPKLLIYWASIRHTGVPSRFSGSGSGTEFTLTISGLQPEDFATYYCQQYSNYPLTFGGGTKLEIKRGGGGSGGGGSGGGGSGGGGSDVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSSD

SEQ ID NO: 74 - protein, 248 aa, Artificial Sequence
  VL1F-linker-VH1H
DIQLTQSHRFLSASVGDRVTITCRASQGVGSAIAWYQQKPGQSPKLLIYWASIRHTGVPSRFSGSGSGTDFTLTISSLQPEDVATYFCQQYSNYPLTFGGGTKVEIKRGGGGSGGGGSGGGGSGGGGSDVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSSD

SEQ ID NO: 75 - protein, 248 aa, Artificial Sequence
  VL1G-linker-VH1H
DIQMTQSPSSLSASVGDRITITCKASQGVGSAIAWYQQKPGRVPKLLIYWASIRHTGVPSRFSGSGSGTEFTLTISSLQAEDVATYYCQQYSNYPLTFGGGTKVEIKRGGGGSGGGGSGGGGSGGGGSDVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSSD

SEQ ID NO: 76 - protein, 248 aa, Artificial Sequence
  VL1H-linker-VH1H
AIQLTQSHKFLSASVGDRVTITCRASQGVGSAIAWYQQKPGQSPKLLIYWASIRHSGVPSRFSGSGSGTDFTLTISSLQSEDFADYFCQQYSNYPLTFGGGTKLEIKRGGGGSGGGGSGGGGSGGGGSDVQLQESGPGLVKPSQTLSLTCTVSGDSITSGYWNWIRKFPGNKLEYIGYISYSDTTYYNPSLKSRVTISVDTSKNQFSLKLKSVTAEDTASYYCASAYYGSSLSMGNWGQGTSATVSSD

SEQ ID NO: 77 - protein, 256 aa, Artificial Sequence
  VL3A-linker-VH3A
DIVLTQSPDSLAVSLGERATISCRASKSVSTGYSYLHWYQQKPGQPPKLLIYLASNLESGVPARFSGSGSGTDFTLTISSLQAEDVAVYYCQHSRELPLTFGAGTKLEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 78 - protein, 256 aa, Artificial Sequence
  VL3A-LINKER-VH3B
DIVLTQSPDSLAVSLGERATISCRASKSVSTGYSYLHWYQQKPGQPPKLLIYLASNLESGVPARFSGSGSGTDFTLTISSLQAEDVAVYYCQHSRELPLTFGAGTKLEIKRGGGGSGGGGSGGGGSGGGGSEVQLQQSGAEVKKPGASVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 79 - protein, 256 aa, Artificial Sequence
  VL3A-linker-VH3C
DIVLTQSPDSLAVSLGERATISCRASKSVSTGYSYLHWYQQKPGQPPKLLIYLASNLESGVPARFSGSGSGTDFTLTISSLQAEDVAVYYCQHSRELPLTFGAGTKLEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTLVTVSS

SEQ ID NO: 80 - protein, 256 aa, Artificial Sequence
  VL3B-linker-VH3A
DIVLTQSPATLSLSPGERATLSCRASKSVSTGYSYLHWYQQKPGQPPKLLIYLASNLESGVPARFSGSGSGTDFTLTISSLEPEDFAVYYCQHSRELPLTFGAGTKLEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 81 - protein, 256 aa, Artificial Sequence
  VL3B-linker-VH3B
DIVLTQSPATLSLSPGERATLSCRASKSVSTGYSYLHWYQQKPGQPPKLLIYLASNLESGVPARFSGSGSGTDFTLTISSLEPEDFAVYYCQHSRELPLTFGAGTKLEIKRGGGGSGGGGSGGGGSGGGGSEVQLQQSGAEVKKPGASVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 82 - protein, 256 aa, Artificial Sequence
  VL3B-linker-VH3C
DIVLTQSPATLSLSPGERATLSCRASKSVSTGYSYLHWYQQKPGQPPKLLIYLASNLESGVPARFSGSGSGTDFTLTISSLEPEDFAVYYCQHSRELPLTFGAGTKLEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTLVTVSS

SEQ ID NO: 83 - protein, 256 aa, Artificial Sequence
  VL3C-linker-VH3A
DIVLTQSPATLSVSPGERATLSCRASKSVSTGYSYLHWYQQKPGQPPKLLIYLASNLESGVPARFSGSGSGTDFTLTISSLQSEDFAVYYCQHSRELPLTFGAGTKVEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 84 - protein, 256 aa, Artificial Sequence
  VL3C-linker-VH3B
DIVLTQSPATLSVSPGERATLSCRASKSVSTGYSYLHWYQQKPGQPPKLLIYLASNLESGVPARFSGSGSGTDFTLTISSLQSEDFAVYYCQHSRELPLTFGAGTKVEIKRGGGGSGGGGSGGGGSGGGGSEVQLQQSGAEVKKPGASVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTTVTVSS

SEQ ID NO: 85 - protein, 256 aa, Artificial Sequence
  VL3C-linker-VHC
DIVLTQSPATLSVSPGERATLSCRASKSVSTGYSYLHWYQQKPGQPPKLLIYLASNLESGVPARFSGSGSGTDFTLTISSLQSEDFAVYYCQHSRELPLTFGAGTKVEIKRGGGGSGGGGSGGGGSGGGGSEVQLQQSGAEVKKPGSSVKVSCKASGYTFTDYNMDWVRQAPGKSLEWIGDINPNYDSTRYNQKFQGRATLTVDKSSSTAYMELSSLRSEDTAVYYCARGDYYVSSYGHDYAMDYWGQGTLVTVSS

SEQ ID NO: 86 - protein, 257 aa, Artificial Sequence
  VL3D-linker-VH3D
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIKRGGGGSGGGGSGGGGSGGGGSQVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSSD

SEQ ID NO: 87 - protein, 257 aa, Artificial Sequence
  VL3E-linker-VH3D
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIKRGGGGSGGGGSGGGGSGGGGSQVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSSD

SEQ ID NO: 88 - protein, 257 aa, Artificial Sequence
  VL3F-linker-VH3D
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIKRGGGGSGGGGSGGGGSGGGGSQVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSSD

SEQ ID NO: 89 - protein, 257 aa, Artificial Sequence
  VL3G-linker-VH3D
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIKRGGGGSGGGGSGGGGSGGGGSQVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSSD

SEQ ID NO: 90 - protein, 257 aa, Artificial Sequence
  VL3H-linker-VH3D
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELKRGGGGSGGGGSGGGGSGGGGSQVQLVQSGAEVTKPGASVKVSCKASGYTFTDYNMDWVRQAPGQGLEWVGDINPNYDSTRYNQKFKGRVTLTRDTSTTTVYMELSSLRSEDTAVYYCARGDYYVSSYGHDYAMDYWGQGTTVTVSSD

SEQ ID NO: 91 - protein, 257 aa, Artificial Sequence
  VL3D-linker-VH3E
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIKRGGGGSGGGGSGGGGSGGGGSQVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSSD

SEQ ID NO: 92 - protein, 257 aa, Artificial Sequence
  VL3E-linker-VH3E
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIKRGGGGSGGGGSGGGGSGGGGSQVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSSD

SEQ ID NO: 93 - protein, 257 aa, Artificial Sequence
  VL3F-linker-VH3E
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIKRGGGGSGGGGSGGGGSGGGGSQVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSSD

SEQ ID NO: 94 - protein, 257 aa, Artificial Sequence
  VL3G-linker-VH3E
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIKRGGGGSGGGGSGGGGSGGGGSQVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSSD

SEQ ID NO: 95 - protein, 257 aa, Artificial Sequence
  VL3H-linker-VH3E
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELKRGGGGSGGGGSGGGGSGGGGSQVQLVESGAEVKKPGASVKLSCKASGYTFTDYNMDWVRQAPGQRLEWMGDINPNYDSTRYNQKFKGRVTITVDKSASTAYMELSSLRSEDTAVYYCARGDYYVSSYGHDYAMDYWGPGTTVTVSSD

SEQ ID NO: 96 - protein, 257 aa, Artificial Sequence
  VL3D-linker-VH3F
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIKRGGGGSGGGGSGGGGSGGGGSQVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSSD

SEQ ID NO: 97 - protein, 257 aa, Artificial Sequence
  VL3E-linker-VH3F
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIKRGGGGSGGGGSGGGGSGGGGSQVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSSD

SEQ ID NO: 98 - protein, 257 aa, Artificial Sequence
  VL3F-linker-VH3F
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIKRGGGGSGGGGSGGGGSGGGGSQVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSSD

SEQ ID NO: 99 - protein, 257 aa, Artificial Sequence
  VL3G-linker-VH3F
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIKRGGGGSGGGGSGGGGSGGGGSQVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSSD

SEQ ID NO: 100 - protein, 257 aa, Artificial Sequence
  VL3H-linker-VH3F
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELKRGGGGSGGGGSGGGGSGGGGSQVQLVQSGAEVKKPGASVKVSCKASGYTFTDYNMDWVRQAPGQGLEWIGDINPNYDSTRYNQKFKGWVTVTRDTSISTAYMELSRLTSDDTAVYYCARGDYYVSSYGHDYAMDYWGQGTMVTVSSD

SEQ ID NO: 101 - protein, 257 aa, Artificial Sequence
  VL3D-linker-VH3G
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIKRGGGGSGGGGSGGGGSGGGGSQVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSSD

SEQ ID NO: 102 - protein, 257 aa, Artificial Sequence
  VL3E-linker-VH3G
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIKRGGGGSGGGGSGGGGSGGGGSQVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSSD

SEQ ID NO: 103 - protein, 257 aa, Artificial Sequence
  VL3F-linker-VH3G
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIKRGGGGSGGGGSGGGGSGGGGSQVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSSD

SEQ ID NO: 104 - protein, 257 aa, Artificial Sequence
  VL3G-linker-VH3G
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIKRGGGGSGGGGSGGGGSGGGGSQVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSSD

SEQ ID NO: 105 - protein, 257 aa, Artificial Sequence
  VL3H-linker-VH3G
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELKRGGGGSGGGGSGGGGSGGGGSQVQLVQSGAEVKKPGASVKVSCKASGYTFTDYNMDWVRQAPGQGLEWMGDINPNYDSTRYNQKFKGRVTSTRDTSISTAYMELSRLRSDDTVVYYCARGDYYVSSYGHDYAMDYWGQGTSVTVSSD

SEQ ID NO: 106 - protein, 257 aa, Artificial Sequence
  VL3D-linker-VH3H
EIVLTQSPSSLSAYVGDRVTITCRASKSVSTGYSYLHWYQQKPGKAPKLLIYLASNLESGVPSRFSGSGSGTDFTLTINSLQPEDFATYYCQHSRELPLTFGGGTKLEIKRGGGGSGGGGSGGGGSGGGGSEVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSSD

SEQ ID NO: 107 - protein, 257 aa, Artificial Sequence
  VL3E-linker-VH3H
DIVLTQSPSSLSASVGDRVTITCRASKSVSTGYSYLHWYQQKPGKAPKLLIYLASNLESGVPSRFSGSGSGTDFTLTISSLQPEDFATYYCQHSRELPLTFGQGTKLEIKRGGGGSGGGGSGGGGSGGGGSEVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSSD

SEQ ID NO: 108 - protein, 257 aa, Artificial Sequence
  VL3F-linker-VH3H
EIVLTQSPATLSLSPGERATLSCRASKSVSTGYSYLHWYQQKPGQAPRLLIYLASNLESGVPARFSGSGSGTDFTLTISSLEPEDFAVYYCQHSRELPLTFGGGTKVEIKRGGGGSGGGGSGGGGSGGGGSEVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSSD

SEQ ID NO: 109 - protein, 257 aa, Artificial Sequence
  VL3G-linker-VH3H
DIQMTQSPSSLSASVGDRVTISCRASKSVSTGYSYLHWYQQKPGQAPKLLIYLASNLESGVPSRFSGSGSGTDFTLTISSLQPEDVATYYCQHSRELPLTFGQGTKVEIKRGGGGSGGGGSGGGGSGGGGSEVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSSD

SEQ ID NO: 110 - protein, 257 aa, Artificial Sequence
  VL3H-linker-VH3H
EIVLTQSPATLSLSIGQRATLSCRASKSVSTGYSYLHWYQQKPGQPPRLLIYLASNLESGIPARFSGSGPGTDFTLTIRPLEDEDFAVYYCQHSRELPLTFGAGTKLELKRGGGGSGGGGSGGGGSGGGGSEVQLVQSGAELVKPGASVKVSCKASGYTFTDYNMDWVRQSHGRGLEWMGDINPNYDSTRYNQKFKGRVTSTVDKSISTAYMELSRLTSDDTVVYYCARGDYYVSSYGHDYAMDYWGQGTSVTVSSD

SEQ ID NO: 111 - protein, 108 aa, Artificial Sequence
  murine parent VL TP1
DIVMTQSHKFMSTSVGDRVSITCKASQGVGSAIAWYQQKTGQSPKLLIYWASIRHTGVPDRFTGSGSGTDFTLTISNVQSEDLADYFCQQYSNYPLTFGGGTKLEIKR

SEQ ID NO: 112 - protein, 120 aa, Artificial Sequence
  murine parent VH TP1
EVQLQESGPSLVKPSQTLSLTCSVTGDSITSGYWNWIRKFPGNKLEYMGYISYSDTTYYNPSLKSRISITRDTSKNQYYLHLKSVTTEDTATYYCASAYYGSSLSMGNWGQGTSATVSSD

SEQ ID NO: 113 - protein, 110 aa, Artificial Sequence
  murine parent VL TP3
DIVLTQSPASLAVSLGQRATISCRASKSVSTGYSYLHWYQQKPGQPPKLLIYLASNLESGVPARFSGSGSGTDFTLNIHPVEEEDAATYYCQHSRELPLTFGAGTKLELK

SEQ ID NO: 114 - protein, 125 aa, Artificial Sequence
  murine parent VH TP3
EVQLQQSGAELVKPGASVKISCKASGYTFTDYNMDWVKQSHGKSLEWIGDINPNYDSTRYNQKFKGKATLTVDKSSSTAYMELRSLTSEDTAVYYCARGDYYVSSYGHDYAMDYWGQGTSVTVSS

SEQ ID NO: 115 - protein, 20 aa, Artificial Sequence
  Leader peptide
METDTLLLWVLLLWVPGSTG